(19) United States
(12) Patent Application Publication
(10) Pub. No.: US 2026/0248952 A1
(43) Pub. Date: Aug. 27, 2026

(54) LIPID MICROBUBBLES FOR THE TARGETED DELIVERY OF ACTIVE AGENTS

(71) Applicants: Centre National de la Recherche Scientifique, PARIS (FR); Université d'Orléans, Orléans (FR)

(72) Inventors: Anthony DELALANDE, SANDILLON (FR); Chantal PICHON, SAINT DENIS DE L'HOTEL (FR)

(21) Appl. No.: 18/872,702

(22) PCT Filed: Jun. 8, 2023
(86) PCT No.: PCT/FR2023/050819

§ 371 Date: Dec. 6, 2024

(30) Foreign Application Priority Data
Jun. 8, 2022 (FR) 2205492

(51) Int. Cl. A61K 47/69; A61K 41/00; A61K 47/54; A61K 47/59; A61K 48/00; C12N 15/88

(52) U.S. Cl. CPC A61K 47/6925; A61K 41/0023; A61K 41/0028; A61K 47/543; A61K 47/59; A61K 48/0041; C12N 15/88

(57) ABSTRACT

The present invention concerns optimized microbubbles, in particular lipid microbubbles, which can be used in the prevention and treatment of diseases or for marking.

The invention relates in particular to a lipid microbubble, comprising at least one cationic compound selected from lipophosphoramidates, histidylated polyethylenimines, and any mixture thereof. The microbubble preferably further comprises at least one agent selected from a therapeutic agent, a targeting agent, a marking agent, and any combination thereof.

The invention also relates to a method for producing this microbubble.

The present invention also relates to a composition, in particular a pharmaceutical composition, and a kit, comprising at least one of these microbubbles. The present invention also relates to the use of these microbubbles, composition, kit, as a medicament or as a marking agent.

### CROSS-REFERENCE TO RELATED APPLICATIONS

The present application is a filing under 35 U.S.C. 371 as the National Stage of International Application No. PCT/FR2023/050819, filed Jun. 8, 2023, entitled “LIPID MICROBUBBLES FOR THE TARGETED DELIVERY OF ACTIVE AGENTS,” which claims priority to French Application No. 2205492 filed with the Intellectual Property Office of France on Jun. 8, 2022, and entitled “LIPID MICROBUBBLES FOR THE TARGETED DELIVERY OF ACTIVE AGENTS,” both of which are incorporated herein by reference in their entirety for all purposes.

### TECHNICAL FIELD OF THE INVENTION

The present invention belongs to the field of targeted delivery of active ingredients, for therapy and/or labeling, as well as to the field of microbubbles, especially functionalized microbubbles.

The present invention relates in particular to optimized microbubbles, especially lipid microbubbles, which can be used for disease prevention, treatment and marking.

The invention relates in particular to a lipid microbubble, comprising at least one cationic compound selected from lipophosphoramidates, histidylated polyethylenimines, and any mixture thereof. The microbubble preferably further comprises at least one agent selected from a therapeutic agent, a targeting agent, a marking agent, and any combination thereof.

The invention also relates to a method for producing this microbubble.

The present invention also relates to a composition, in particular a pharmaceutical composition, and a kit, comprising at least one of these microbubbles. The present invention also relates to the use of these microbubbles, composition, kit, as a medicament or as a marking agent.

### STATE OF THE ART

Initially developed for diagnostic purposes, microbubbles (MB) can also be used as vectors for therapeutic purposes. Indeed, thanks to the wide variety of molecules that can make up the envelope, it is possible to encapsulate drugs or to complex nucleic acids. In general, MB used as vectors have a functionable lipid envelope, enabling therapeutic molecules to be either embedded between the lipids or dissolved in a drop of oil inside the envelope. In addition, using cationic lipids, nucleic acids can be complexed by electrostatic interactions. Other lipids can also be used to attach nanoparticles or antibodies, using streptavidin-biotin interactions, or functionalized for chemistry-click applications.

A few studies have led to the creation of MB as vectors for molecules of interest. Zhu et al., Scientific Reports, 2016, describe MB loaded with a chemotherapeutic agent (paclitaxel). Fan et al, Biomaterials, 2013, reported the development of microbubbles loaded with an antineoplastic agent (1,3-bis(2-chloroethyl)-1-nitrosourea or BCNU). However, the drugs complexed to these MB are limited to anti-cancer chemical compounds. In particular, the possibility of delivering nucleic acids with these microbubbles has not been demonstrated. However, an increasing number of emerging therapies, notably anti-cancer therapies, use nucleic acids. These molecules offer a flexibility unmatched by chemical compounds, enabling personalized therapies, for example.

Delalande et al, Bioscience Reports, 2017, describe cationic MB complexed to plasmid DNAs. However, the nucleic acid delivery efficiency and targeting specificity of these MB remains limited. These aspects are particularly critical in the treatment of brain pathologies. In addition, one of the major obstacles to treatment concerns crossing the blood-brain barrier (BBB). For these reasons, molecules to be delivered to the central nervous system have traditionally been selected for their ability to passively cross the BBB. This implies low nucleic acid delivery efficiency, as well as a virtual absence of targeting, which can be the cause of numerous harmful side-effects.

It is therefore essential to develop new, effective and specific ways of delivering therapeutic agents to the brain, particularly nucleic acids able to cross the BBB without degradation.

In this context, microbubbles represent a promising system. For example, Fan et al, 2016, have produced folate containing MB capable of complexing nucleic acid for targeted brain transfection. These cationic microbubbles are based on the use of DSTAP or DPTAP lipids, using a tri-methyl ammonium function as the cationic charge for complexation. The main disadvantage of these formulations is that they are not sufficiently effective to be used in gene therapy protocols. In addition, these formulations have been shown to be toxic.

Thus, there's still a need to develop microbubbles capable of efficiently and specifically delivering therapeutic agents, such as nucleic acids, to specific organs or even cells.

The present invention makes it possible to meet this need, by describing microbubbles based on new cationic formulations whose advantages are as follows:
Better nucleic acid compaction,
Lower toxicity,
Use of the endosomal escape system (“proton sponge effect”) to increase transfection and avoid the lysosomal pathway,
Improved transfection efficiency.

Thus, the microbubbles developed by the present Inventors have the ability to transport drugs, notably nucleic acids, stably into the bloodstream; to actively cross the blood-brain barrier; to deliver drugs in a targeted manner, notably towards antigens of interest. The technology developed here makes it possible to transiently open the BBB and efficiently deliver nucleic acid-type active ingredients across it. It also enables vessels to be permeabilized, so that molecules of interest can be delivered.

### DESCRIPTION OF THE INVENTION

In the context of the present invention, the Inventors have developed innovative microbubbles, in particular innovative lipid microbubbles, capable of stably transporting drugs, in particular nucleic acids, into the bloodstream; actively crossing the blood-brain barrier (BBB); and delivering drugs in a targeted manner, in particular towards antigens of interest.

In particular, the Inventors have shown that, surprisingly, the lipid microbubbles developed this way possess significantly improved stability, unlike the microbubbles described in the prior art. Remarkably, the data also reveals that these optimized microbubbles are able to deliver agents of interest, notably nucleic acids, more efficiently than microbubbles described in the prior art. In particular, these microbubbles are capable of actively crossing vessels, as well as the BBB or tumor microenvironment. The Inventors have also demonstrated that the localized application of ultrasound enables these optimized microbubbles to be targeted very precisely to the area to be treated, including very difficult-to-access areas such as the central nervous system, vessels, and the tumor microenvironment. These data thus reveal the therapeutic potential of these lipid microbubbles to treat a wide range of pathologies in a targeted manner, including central nervous system pathologies, vessel pathologies, tumors, cancers. The data also show that these optimized microbubbles are detection and imaging tools.

The present invention therefore provides both powerful, broad-spectrum treatment methods for pathologies, as well as efficient and reliable diagnostic methods.

### DETAILED DESCRIPTION OF THE INVENTION

### Summary of the Invention

The present invention relates in particular to lipid microbubbles, which can be used both in the prevention and treatment of pathologies, and in detection and imaging, particularly medical.

The present invention relates in particular to a lipid microbubble, comprising at least one cationic compound selected from lipophosphoramidates, histidylated polyethylenimines, and any mixture thereof.

The present invention also relates to a pharmaceutical composition comprising at least one microbubble as defined above, and, optionally, a pharmaceutically acceptable excipient, the concentration of microbubbles in the composition preferably ranging from 10⁶ to 10¹⁴ microbubbles/ml, more preferably from 10⁷ to 10¹³ microbubbles/ml, more preferably from 10⁸ to 10¹² microbubbles/ml, more preferably from 10⁹ to 10¹¹ microbubbles/ml, more preferably the concentration of microbubbles in the composition being about 10¹⁰ microbubbles/ml.

The present invention also relates to a kit, comprising:
a) at least one microbubble as defined above, in a first container;
b) at least one therapeutic agent, in a second container;
c) optionally, at least one targeting agent in a third container;
d) optionally, at least one marking agent in a fourth container;
e) optionally, instructions for preparation and/or use;
   the therapeutic agent and/or the targeting agent and/or the marking agent being preferably selected from:
1) a nucleic acid;
2) a liposoluble active ingredient;
3) a chemotherapeutic agent, such as a cytotoxic agent and/or a cytostatic agent;
4) an antibody;
5) a protein;
6) an antigen;
7) a toxin;
8) a receiver
9) an enzyme;
10) a hormone;
11) a ligand;
12) a viral vector;
13) a nanoparticle, preferably comprising at least one agent selected from a therapeutic agent, a targeting agent, a marking agent, and any combination thereof;
14) any derivative of 1) to 13), preferably any functional derivative thereof;
15) any fragment from 1) to 14), preferably any functional fragment thereof; and
16) any combination of 1) to 15);

More preferably from a nucleic acid, a liposoluble active ingredient, a chemotherapeutic agent, an antibody, an antibody derivative, a functional fragment of an antibody or its derivative, a protein, a protein fragment, a nanoparticle, and any combination thereof.

According to another aspect, the present invention relates to a microbubble, a pharmaceutical composition, or a kit, as defined above, for use as a medicament or as a marking agent. According to another aspect, the present invention relates to a method for producing at least one microbubble as defined above, comprising the following steps:
a) A mixture, in a container, of cationic compounds selected from lipophosphoramidates, histidylated polyethylenimines, and any mixtures thereof; ethanol; and optionally at least one agent selected from a therapeutic agent, a targeting agent, a marking agent, and any combination thereof;
b) Evaporation of the mixture obtained in step a) to obtain a lipid film and rehydration of the lipid film to form a liposomal suspension; this entire step can also be performed using microfluidics;
c) Lyophilization of the liposomal suspension obtained in step b);
d) Replacement of the air contained in the container containing the lyophilizate obtained in step c), by a biocompatible gas, the gas preferably being selected from perfluorobutane (C4F10), perfluoropropane (C3F8), dinitrogen (N2), sulfur hexafluoride (SF6), nitrogen oxide (NO), hydrogen, dioxygen, helium, xenon, argon, nitrous oxide (N2O), and any mixture thereof;
e) Rehydration of the lyophilizate from step d) to obtain a solution;
f) Stirring the solution obtained in step d) to form microbubbles;
g) Optionally, functionalization of the microbubbles with at least one agent selected from a therapeutic agent, a targeting agent, a marking agent, and any combination thereof; and/or functionalization of the microbubbles with the addition of at least one functional group enabling binding to at least one agent selected from a therapeutic agent, a targeting agent, a marking agent, and any combination thereof.

### Definitions

The terms “at least one” are here considered synonymous with the terms “one or more”.

As used herein, the term “microbubble” refers to a bubble whose diameter ranges from about one micrometer to several tens of micrometers (up to about one hundred micrometers), typically from about one micrometer to about ten micrometers. A microbubble comprises an envelope surrounding a filler material. The envelope may consist of lipids, proteins, sugars, ionic compounds, or mixtures thereof. In the case of an envelope made of lipids or essentially made of lipids, we speak of a lipid microbubble.

Among the compounds that can be used to form a microbubble envelope according to the present invention are, in particular:
Cationic compounds, such as lipophosphoramidates, histidylated polyethylenimines, and mixtures thereof; and/or
lipids, in particular selected from the group consisting of dimyristoyl-glycero-phosphocholine, distearoyl-glycero-phosphocholine, dimyristoyl-glycero-phosphoethanolamine-polyethylene glycol, distearoyl-glycero-phosphoethanolamine-polyethylene glycol 2000, distearoyl-glycero-phosphoethanolamine-[biotinyl (polyethylene glycol)], cholesterol, beta-sitosterol, 1,2-dioleoyl-sn-glycero-3-phosphoethanolamine (DOPE), 1-oleoyl-2-[6-[(7-nitro-2-1,3-benzoxadiazol-4-yl)amino]hexanoyl]-3-trimethylammonium propane (DOTAP), 1,2-dimyristoyl-rac-glycero-3-methoxypolyethylene glycol-2000 (DMG-PEG2000), clickable lipids DBCO, Tetrazine, Methyl tetrazine, NHS (DSPE-PEG2000-X), and any combination thereof;
and any combination thereof.

The filler material of the microbubble can be a gas of any type. The filling gas is advantageously biocompatible (i.e. it is well tolerated by a living organism). In particular, the filling gas can be selected from perfluorobutane (C4F10), perfluoropropane (C3F8), dinitrogen (N2), sulfur hexafluoride (SF6), nitrogen oxide (NO), hydrogen, dioxygen, helium, xenon, argon, nitrous oxide (N2O), and any mixture thereof.

By “lipid microbubble” we mean a microbubble whose envelope is essentially made up of lipids, or is made up of lipids. The term “envelope essentially made up of lipids” here means an envelope comprising 55% lipids or more, preferably 60% lipids or more, preferably 70% lipids or more, preferably 80% lipids or more, preferably 90% lipids or more, preferably 91% lipids or more, preferably 92% lipids or more, preferably 93% of lipids or more, preferably 94% of lipids or more, preferably 95% of lipids or more, preferably 96% of lipids or more, preferably 97% of lipids or more, preferably 98% of lipids or more, even more preferably 99% of lipids or more, the percentage being expressed in lipid mass relative to the total mass of the envelope.

Advantageously, the lipid microbubble comprises at least one cationic compound, in particular a cationic lipid or a lipid coupled to at least one cationic polymer.

The envelope of the lipid microbubble may comprise a cationic lipid or a lipid coupled to at least one cationic polymer, or consist essentially of cationic lipids or lipids coupled to at least one cationic polymer, or consist of cationic lipids and/or lipids coupled to at least one cationic polymer. Advantageously, the envelope comprises cationic lipids and/or lipids coupled to at least one cationic polymer, and fusogenic lipids.

According to an implementation, the lipid envelope of the microbubble comprises from 2 to 50% of cationic lipids, preferably at least 2% of cationic lipids, preferably at least 10% of cationic lipids, preferably at least 20% of cationic lipids, preferably at least 30% of cationic lipids, preferably at least 40% of cationic lipids, preferably 50% of cationic lipids, the percentage being expressed in moles of cationic lipids with respect to the number of moles of total lipids constituting the envelope.

By “cationic compound” we mean a compound comprising at least one cation and whose overall charge in solution is positive. Examples of cationic compounds (cationic lipids) that can be used to form a microbubble envelope include, without limitation: lipophosphoramidates, histidylated polyethylenimines, and any mixture thereof. By “cationic lipid” we mean a lipid comprising at least one cation and whose overall charge in solution is positive.

By “lipophosphoramidate” we mean a bioinspired amphiphilic phospholipid that carries a cationic charge on its polar head.

Lipophosphoramidates include dimyristoyl phosphoramidates (such as dimyristoyl bromide phosphoramidates, and dimyristoyl histamine phosphoramidates), dioleyl phosphoramidates (such as dioleyl methylimidazolium phosphoramidates), dipalmitoyl phosphoramidates, and disteraoyl phosphoramidates.

By “polyethylenimine”, or “polyethyleneimine”, or “PEI”, or “polyaziridine” we mean an organic polymer of the chemical formula H[CH₂—CH₂—NH—]ₙH. By “histidylated polyethylenimine” is meant a polyethylenimine comprising at least one histidyl group. Advantageously, the histidylated polyethylenimine used to form the microbubble is coupled to a fatty acid. Examples of fatty acids that can be coupled to a histidylated polyethylenimine include stearic acid, myristic acid, palmitic acid, oleic acid and any combination thereof.

By “compound/agent exposed to the microbubble surface” we mean a compound or agent conjugated/coupled/bonded to the external surface of the microbubble (e.g. conjugated/coupled/bonded to the microbubble envelope) and in contact with the medium outside the microbubble.

By “compound/agent embedded in the lipid envelope of the microbubble”, we mean a compound or agent partially or totally integrated/incorporated in the layer of compounds (in particular lipids) forming the microbubble envelope. Thus, a compound or agent totally integrated/incorporated into the envelope is in contact only with the compounds (in particular lipids) forming the microbubble envelope: in this case, the compound or agent is in contact neither with the environment outside, nor with the environment inside, the microbubble. On the other hand, a compound or agent partially integrated/incorporated into the envelope can be in contact either with the outside medium of the microbubble, or with the inside medium of the microbubble, or with both the outside and the inside medium of the microbubble (“through-compound”).

By “compound/agent incorporated inside the microbubble”, we mean a compound or agent located in the inside medium of the microbubble (i.e. in the medium/cavity formed by the microbubble envelope). This compound or agent may be in contact with the inner surface of the microbubble (e.g. the inner surface of the microbubble envelope). In particular, it may be conjugated/coupled/bonded to the inner surface of the microbubble (e.g. the inner surface of the microbubble envelope).

By “Therapeutic agent” or “therapeutic compound”, we mean any agent, compound or molecule presented as having curative or preventive properties in respect of human or animal pathologies or diseases. A therapeutic agent or compound therefore includes any agent or compound that can be used in or administered to humans or animals with the aim of establishing a medical diagnosis or restoring, correcting or modifying their physiological functions by exerting a pharmacological, immunological and/or metabolic action. The therapeutic agent may therefore be a pharmacological agent.

The therapeutic agent or compound can be of any nature or type and does not depend on its origin. The therapeutic agent may be chemically synthesized, naturally occurring, recombinantly produced (and optionally purified), or synthetically designed and produced. In particular, it may be a small molecule, a nucleic acid, a peptide (including a post-translationally modified peptide), a polypeptide (including a post-translationally modified polypeptide), a protein (including a post-translationally modified protein), a chemical compound, a cancer chemotherapy agent (such as a cytostatic or cytological agent), an antibody, a toxin, an antigen, a hormone, an enzyme, a ligand, a receptor, an antiviral compound, an antibiotic compound, an antifungal compound, an antibacterial compound, a nanoparticle, or any fragment thereof (preferably a functionally active fragment), or any derivative thereof (preferably a functionally active derivative), such as a peptidomimetic, a mimetic antibody, a chemical derivative, among others. The therapeutic agent or compound may, for example, comprise, or consist essentially of, or consist of, a nucleic acid, a lipid-soluble active, a chemotherapeutic agent (such as a cytotoxic agent and/or a cytostatic agent), an antibody, an antibody derivative, a functional fragment of an antibody or antibody derivative, a protein (including a post-translationally modified protein), a protein fragment (such as a peptide, an antigen, an epitope, a functional protein domain, and any combination thereof; including a post-translationally modified protein fragment), a nanoparticle, etc.

By “targeting agent” or “targeting compound”, we mean any agent, compound or molecule presented as being capable of recognizing and/or binding to another molecule (known as the “target molecule”), preferably in a specific way. These terms therefore also include “binding agents” or “binding compounds”. The terms “binding agents” or “binding compounds” refer to any agent or compound or molecule capable of binding to another molecule (known as a “target molecule”), said binding preferably being a specific binding.

The targeting/binding agent recognizes a defined site, domain, region, pocket, epitope, spatial configuration, conformation, chemical grouping, or any combination thereof, of the target molecule. The targeting/binding agent can be of any nature or type and is not dependent on its origin. The targeting/binding agent can be chemically synthesized, naturally occurring, recombinantly produced (and optionally purified), or synthetically designed and produced. In particular, it may be a small molecule, a nucleic acid, a peptide (including a post-translationally modified peptide), a polypeptide (including a post-translationally modified polypeptide), a protein (including a post-translationally modified protein), a chemical compound, an antibody, a toxin, an antigen, an epitope, a hormone, an enzyme, a ligand, a receptor, a nanoparticle, or any fragment thereof (preferably a functionally active fragment), or any derivative thereof (preferably a functionally active derivative), such as a peptidomimetic, a mimetic antibody, a chemical derivative, among others, and so on. The targeting/binding agent may for example comprise, or consist essentially of, or consist of, a nucleic acid, a liposoluble active, a chemotherapeutic agent (such as a cytotoxic agent and/or a cytostatic agent), an antibody, an antibody derivative, a functional fragment of an antibody or antibody derivative, a protein (including a post-translationally modified protein), a protein fragment (such as a peptide, an antigen, an epitope, a functional protein domain, and any combination thereof; including a post-translationally modified protein fragment), a nanoparticle, etc.

The target molecule can be of any nature or type and does not depend on its origin. In particular, the target molecule can be a pathogen (such as a virus, bacteria, parasite, etc.) or a fragment thereof (e.g. a nucleic acid, protein, polypeptide, peptide, epitope, lipid, sugar, etc.).

By “marking agent” or “marker” we mean any agent or compound, or molecule presented as being capable of marking another molecule (preferably in a specific way) and being detected by any means. The means of detecting the marking agent are well known to the person skilled in the art, who is perfectly capable of selecting the appropriate technique depending on the marking agent used. Means of marking agent detection include, but are not limited to, the following techniques: optical detection techniques (such as techniques using fluorescence, absorbance, diffraction, light scattering, interferometry, reflectometry, ellipsometry, surface plasmon resonance (SPR), spectroscopy, magnetic particles, etc.), mechanical detection techniques (e.g., optical detection of the marking agent), electrical detection techniques (such as techniques using electrodes, electrical sensors, etc.). The marking agent can therefore be a contrast agent.

The targeting/binding agent can be of any nature or type and does not depend on its origin. The targeting/binding agent can be chemically synthesized, naturally occurring, recombinantly produced (and optionally purified), or synthetically designed and produced. In particular, it may be a small molecule, a nucleic acid, a peptide (including a post-translationally modified peptide), a polypeptide (including a post-translationally modified polypeptide), a protein (including a post-translationally modified protein), a chemical compound, an antibody, a toxin, an antigen, an epitope, a hormone, an enzyme, a ligand, a receptor, a nanoparticle, or any fragment thereof (preferably a functionally active fragment), or any derivative thereof (preferably a functionally active derivative), such as a peptidomimetic, a mimetic antibody, a chemical derivative, among others, and so on. The labeling agent may for example comprise, or consist essentially of, or consist of, a nucleic acid, a lipid-soluble active, a chemotherapeutic agent (such as a cytotoxic agent and/or a cytostatic agent), an antibody, an antibody derivative, a functional fragment of an antibody or antibody derivative, a protein (including a post-translationally modified protein), a protein fragment (such as a peptide, an antigen, an epitope, a functional protein domain, and any combination thereof; including a post-translationally modified protein fragment), a nanoparticle, etc.

The labeling agent may for example comprise, or consist essentially of, a fluorophore (e.g. fluorescein or luciferase), a fluorescent protein/polypeptide/peptide (e.g. GFP and its variants, such as RFP, CFP, YFP, etc.), a radioisotope (particularly suitable for scintigraphy, e.g. ^{99m}Tc), an antibody-recognizable label (e.g. c-Myc protein or a poly-histidine label), an affinity label (e.g. biotin, streptavidin, etc.), an enzyme (e.g. horseradish peroxidase), a contrast agent, a peptide tag, etc.

The term “derivative” is generally used to designate a component or species (protein, antibody, protein fragment, polypeptide, polynucleotide, oligonucleotide, nucleoside, nucleotide, vector, virus, etc.) with one or more modifications compared with a reference component (for example, the wild-type component as found in nature as originally identified, i.e. the corresponding “original” component known as the original component). A derivative may in particular be a fragment, a part, a variant, a mutant, a synthetic version (e.g. manufactured, notably in vitro), a mimetic, or a combination thereof, of the original component or species. The terms “variant”, or “mutant”, can be used interchangeably to refer generally to a component or species (protein, antibody, protein fragment, polypeptide, polynucleotide, oligonucleotide, nucleoside, nucleotide, vector, virus, etc.) presenting one or more modifications in relation to a reference component (for example, the wild-type component as found in nature as originally identified, i.e. the corresponding “original” component called the original component). A nucleotide or nucleoside variant may have a modified base and/or a modified sugar and/or a modified linkage. For polypeptide, polynucleotide and antibody variants, any modification can be envisaged, including substitution, insertion, deletion, and any combination thereof, of one or more nucleotide/amino acid residues. The variant can be of natural or artificial origin (e.g. mutated and/or engineered).

When several mutations are envisaged, they may concern consecutive residues and/or non-consecutive residues. Preferred are variants (for example, respectively, protein variants, peptide variants, antibody variants, virus variants, etc.) which retain a degree of sequence identity of at least 80% with the reference component (for example, respectively, the corresponding “original” protein, the corresponding “original” protein fragment, the corresponding “original” polynucleotide). By way of example, “at least 80% identity” means 80%, 81%, 82%, 83%, 84%, 85%, 86%, 87%, 88%, 89%, 90%, 91%, 92%, 93%, 94%, 95%, 96%, 97%, 98% or 99%. In some embodiments, an identity of at least 80% also encompasses an identity of 100%.

By “functional fragment”, or “functionally active fragment”, we mean any fragment of a molecule, agent, species or compound, exhibiting at least one of the original functions of the molecule, agent, species or compound from which said fragment is derived. Preferably, the functional fragment performs said function with an efficiency equal to at least 30% of that of said peptide or protein, preferably at least 40%, preferably at least 45%, preferably at least 50%, preferably at least 55%, preferably at least 60%, preferably at least 65%, preferably at least 70%, preferably at least 75%, preferably at least 80%, preferably at least 85%, preferably at least 90%, preferably at least 91%, preferably at least 92%, preferably at least 93%, preferably at least 94%, preferably at least 95%, preferably at least 96%, preferably at least 97%, preferably at least 98%, preferably at least 99%, preferably at least 100% of the efficacy of said molecule, said agent, said species, said compound, from which said fragment is derived.

By “identity” or “sequence identity” we mean an exact sequence match between two polypeptides or amino acids, or between two nucleic acid molecules or oligonucleotides. The percentages of identity referred to in the present invention are determined after optimal global alignment of the sequences to be compared, which may therefore include one or more additions, deletions, truncations and/or substitutions. This percentage of identity can be calculated by any sequence analysis method well known to the skilled person. The percentage identity is determined after global alignment of the sequences to be compared, taken as a whole, over their entire length. In addition to the manual method, global sequence alignment can also be determined using the algorithm of Needleman and Wunsch (1970).

In particular, for nucleotide sequences, sequence comparison can be carried out using any software well-known to the skilled person, such as Needle software. The parameters used may include: “Gap Open” equal to 10.0, “Gap Extend” equal to 0.5 and the EDNAFULL matrix (EMBOSS version of NCBI NUC4.4).

Amino acid sequences can be compared using any software program familiar to the skilled person, such as Needle. The parameters used may include “Gap Open” equal to 10.0, “Gap Extend” equal to 0.5 and the BLOSUM62 matrix.

By way of illustration, “at least 80% sequence identity”, as used here, represents in particular 80%, 81%, 82%, 83%, 84%, 85%, 86%, 87%, 88%, 89%, 90%, 91%, 92%, 93%, 94%, 95%, 96%, 97%, 98%, 99% or 100% sequence identity.

The terms “polynucleotide”, “nucleic acid molecule” and “nucleic acid” are used interchangeably herein and refer to a polymeric macromolecule or oligomer made up of nucleotide monomers (preferably at least 5 nucleotide monomers, also known as nucleotide residues). Nucleotide monomers are composed of a nucleobase, a five-carbon sugar (such as, but not limited to, ribose or 2′-deoxyribose), and one to three phosphate groups. Typically, a polynucleotide is formed by phosphodiester bonds between individual nucleotide monomers. Nucleic acid molecules include, but are not limited to, ribonucleic acid (RNA), deoxyribonucleic acid (DNA) and their mixtures, such as RNA-DNA hybrids (mixed polyribo-polydeoxyribonucleotides). These terms include single- or double-stranded, linear or circular, natural or synthetic, unmodified or modified versions thereof (e.g. genetically modified polynucleotides; optimized polynucleotides), sense or antisense polynucleotides, chimeric mixtures (e.g. RNA-DNA hybrids). In addition, a polynucleotide may comprise nucleotides of non-natural origin and may be interrupted by non-nucleotide components. Examples of DNA nucleic acids include, without limitation, complementary DNA (cDNA), genomic DNA, plasmid DNA, DNA vector, viral DNA (e.g. viral genomes, viral vectors), oligonucleotides, probes, primers, satellite DNA, microsatellite DNA, coding DNA, non-coding DNA, antisense DNA, and any mixture thereof. Exemplary RNA nucleic acids include, without limitation, messenger RNA (mRNA), precursor messenger RNA (pre-mRNA), small interfering RNA (siRNA), short hairpin RNA (shRNA), microRNA (miRNA), RNA vector, viral RNA, guide RNA (gRNA), antisense RNA, coding RNA, non-coding RNA, antisense RNA, satellite RNA, small cytoplasmic RNA, small nuclear RNA, etc. The polynucleotides described herein can be synthesized by standard methods known in the art, for example using an automated DNA synthesizer (such as those commercially available from Biosearch, Applied Biosystems, etc.) or obtained from a natural source (e.g. a genome, cDNA, etc.) or an artificial source (such as a commercially available library, plasmid, etc.) using molecular biology techniques well known in the art (e.g. cloning, PCR, etc.). Nucleic acids can, for example, be synthesized chemically, for example according to the phosphotriester method (see, for example, Uhlmann, E. & Peyman, A. (1990) Chemical Reviews, 90, 543-584).

The nucleic acid may comprise at least one modified nucleotide (i.e. a nucleotide which is not a nucleotide of a natural DNA or RNA). In particular, these modified nucleotides can be used to increase the nucleic acid's resistance to degradation by nucleases. This is particularly advantageous for RNAs, which are generally more sensitive to nucleases than DNA aptamers. RNA comprising at least one modified nucleotide is called modified RNA. DNA comprising at least one modified nucleotide is called modified DNA.

The nucleic acid can also comprise at least one additional group, in addition to the nucleotides making up its nucleic acid sequence. In this way, the nucleic acid can be linked to at least one additional group.

By “modified DNA” we mean a DNA containing at least one modified nucleotide. A modified DNA may in particular be a DNA in which the nucleic acid backbone is modified, in whole or in part, in particular to make it resistant to hydrolytic degradation, in particular by the action of nucleases. DNA can be modified in its entirety (i.e. every nucleotide that makes it up is modified) or in part (i.e. only some of the nucleotides that make it up are modified). When DNA is modified in part, we can choose to modify all or part of the purines and/or all or part of the pyrimidines.

By “modified RNA” we mean an RNA comprising at least one modified nucleotide. A modified RNA may in particular be an RNA in which the nucleic acid backbone is modified, in whole or in part, in particular to make it resistant to hydrolytic degradation, in particular by the action of nucleases. RNA can be modified in its entirety (i.e. every nucleotide that constitutes it is modified) or in part (i.e. only some of the nucleotides that constitutes it are modified). When RNA is modified in part, you can choose to modify all or part of the purines and/or all or part of the pyrimidines. Modifications of a DNA or RNA (and/or a nucleotide) are well known to the skilled person and can be chosen from: modification of the OH function on the carbon in position 2′ of the ribose by methylation; substitution of the OH function on the carbon in position 2′ of the ribose by an O-Methoxyethyl group; substitution of the OH function on the carbon in position 2′ of the ribose by an amino group; substitution of the OH function on the 2′-position of the ribose carbon by a halogen (notably fluorine); replacement of the phosphodiester (PO) by a phosphorothioate (PS) group (referred to as a phosphorothioate skeleton); the use of a Locked Nucleic Acid (LNA) structure, i.e. the formation of a methylene bridge to lock the ribose in the C3′-endo (N-type) conformation; the use of a Peptide Nucleic Acid (PNA) structure, i.e. the replacement of the sugar-phosphate skeleton by a peptide-type skeleton; and any combination thereof.

By “vector”, we mean a vehicle, preferably a nucleic acid molecule or a viral particle, which contains the elements necessary to enable the administration, propagation and/or expression of one or more nucleic acid molecules in a host cell or organism.

From a functional point of view, this term encompasses vectors for maintenance (cloning vectors), vectors for expression in various host cells or organisms (expression vectors), extrachromosomal vectors (e.g. multicopy plasmids) or integration vectors (e.g. designed to integrate into the genome of a host cell and produce additional copies of the nucleic acid molecule it contains when the host cell replicates). The term also encompasses shuttle vectors (e.g. operating in both prokaryotic and/or eukaryotic hosts) and transfer vectors (e.g. for transferring nucleic acid molecule(s) into the genome of a host cell).

From a structural point of view, vectors can be natural, synthetic or artificial genetic sources, or a combination of natural and artificial genetic elements.

Thus, in the context of the invention, the term “vector” is to be understood broadly to include plasmid and viral vectors.

A “plasmid” as used here refers to a replicable DNA construct. Typically, plasmid vectors contain selection marker genes that enable host cells carrying the plasmid to be identified and/or selected positively or negatively in the presence of the compound corresponding to the selection marker. A variety of positive or negative selection marker genes are known in the art. By way of illustration, an antibiotic resistance gene can be used as a positive selection marker gene to select a host cell in the presence of the corresponding antibiotic.

The term “viral vector” as used herein refers to a nucleic acid vector that comprises at least one element of a virus genome and can be packaged into a viral particle or virus particle. Viral vectors can be replication-competent or selective (e.g., designed to replicate better or selectively in specific host cells), or can be genetically inactivated so as to be replication-defective or defective.

By “Polypeptide”, “protein”, “protein fragment” and “peptide”, we mean polymers of amino acid residues comprising at least nine amino acids linked by peptide bonds. The polymer may be linear, branched or cyclic. The polymer may comprise natural amino acids and/or amino acid analogues and may be interrupted by non-amino acid residues. As a general indication and without however being bound by it herein, if the amino acid polymer contains more than 50 amino acid residues, it is preferably called a polypeptide or protein, whereas if the polymer consists of 50 or fewer amino acids, it is preferably called a “peptide”. The reading and writing directions of an amino acid sequence of a polypeptide, protein and peptide as used herein are the conventional reading and writing directions. The reading and writing convention for amino acid sequences of a polypeptide, protein and peptide places the amine terminus on the left, the sequence then being written and read from the amine terminus (N-terminus) to the carboxyl terminus (C-terminus), from left to right.

The amino acids making up polypeptides, proteins and peptides include “standard” amino acids (also known as “natural amino acids”, a non-exhaustive list of which is given in Table 1 below), as well as non-standard amino acids (also known as “rare amino acids”, including, for example, pyrrolysine (symbolized by the letter O), selenocysteine (symbolized by the letter U), alloisoleucine, allothreonine, ornithine, etc.).

TABLE 1
Standard amino acids

| Name | 3 letters | 1 letter |
|---|---|---|
| alanine | Ala | A |
| arginine | Arg | R |
| asparagine | Asn | N |
| Aspartate or aspartic acid | Asp | D |
| cysteine | Cys | C |
| Glutamate or glutamic acid | Glu | E |
| glutamine | Gln | Q |
| glycine | Gly | G |
| histidine | His | H |
| isoleucine | Island | I |
| leucine | Leu | L |
| lysine | Lilies | K |
| methionine | Met | M |
| phenylalanine | Phe | F |
| proline | Pro | P |
| serine | Ser | S |
| threonine | Thr | T |
| tryptophan | Trp | W |
| tyrosine | Tyr | Y |
| valine | Val | V |

By “peptide or protein fragment” or “peptide or protein part”, we mean a portion of a peptide or protein, i.e. a portion of the consecutive amino acid sequence making up said peptide or protein (referred to as the peptide or protein from which the fragment is derived). The peptide or protein fragment preferably comprises at least 10 consecutive amino acids of the peptide or protein from which it is derived; even more preferably at least 12 consecutive amino acids, even more preferably at least 15 consecutive amino acids, even more preferably at least 20 consecutive amino acids, even more preferably at least 30 consecutive amino acids of the peptide or protein from which it is derived. The peptide or protein fragment preferably has a three-dimensional structure, under non-denaturing conditions (e.g. conditions that are usually non-denaturing for proteins, notably in the absence of denaturing and/or chaotropic agents).

As used herein, the term “post-translational modification” refers to a chemical or enzymatic modification occurring naturally or unnaturally on a protein or protein fragment, after or concomitantly with protein translation (e.g. biological or biochemical synthesis, using e.g. cellular machinery), or after or concomitantly with protein synthesis (e.g. artificial and/or chemical synthesis). This means that at least one of the natural amino acids of the protein or protein fragment is modified by the addition of at least one chemical group and/or the modification (including, but not limited to, removal) of at least one chemical group of the natural amino acid. Examples of such chemical or enzymatic modifications include, but are not limited to, glycosylation, phosphorylation, acylation, carboxylation, acetylation, biotinylation, hydroxylation, lipoylation, amidation, ubiquitination, sumoylation, deamination and so on. By “post-translationally modified protein”, we mean a protein with at least one post-translational modification. By “post-translationally modified protein fragment”, we mean a protein fragment having at least one post-translational modification.

By “liposoluble active ingredient”, we mean any agent, compound or molecule that solubilizes in a fatty substance, and has a biological activity, in particular a therapeutic, pharmacological, targeting or marking activity, as defined above in relation to therapeutic, targeting or marking agents. Liposoluble active ingredients include those that solubilize in lipids or their derivatives, for example in oils, butters, oily esters and other ingredients comprising lipids or their derivatives.

By “Chemotherapeutic agent”, we mean any agent, compound or molecule with chemotherapeutic activity. Chemotherapeutic agents include agents with anti-cancer activity (in particular agents capable of eliminating cancer cells and/or tumors, and/or inducing/stimulating the elimination of cancer cells and/or tumors), as well as agents with anti-autoimmune disease activity. The chemotherapeutic agent may therefore be a cytotoxic agent and/or a cytostatic agent. Examples of chemotherapeutic agents include, but are not limited to, paclitaxel, doxorubicin, gencitabin (e.g. Gemzar), temozolomide, etc.

By “Antibody”, we mean a protein or glycoprotein belonging to the immunoglobulin superfamily; the terms antibody and immunoglobulin are used interchangeably. In mammals, antibodies are mostly secreted by cells derived from B lymphocytes: plasma cells. In particular, they are used by the immune system to specifically detect and neutralize foreign bodies (especially pathogens such as bacteria, viruses, parasites, etc.). Antibodies also include autoantibodies (produced, for example, in autoimmune diseases). The antibody recognizes a unique part of the foreign target, its antigen.

Antibodies have a structure made up of 4 polypeptide chains (150,000 amu or Dalton): two identical heavy chains (H for “heavy”, 50,000 amu each) and two identical light chains (L for “light”, 25,000 amu each), which are linked by a variable number of disulfide bridges ensuring the molecule's cohesion. These chains form a Y structure (half of each light chain constitutes an arm of the Y) and are made up of immunoglobulin domains of up to 110 amino acids. Each light chain is made up of a constant domain (called CL) and a variable domain (called VL); heavy chains are made up of a variable domain (called VH) and, depending on the isotype, three or four constant domains respectively called CH1, CH2, CH3, (CH4). For a given antibody, the two heavy chains are identical, as are the two light chains. The constant domains are characterized by an amino acid sequence that is very similar from one antibody to the next, characteristic of the species and isotype. Constant domains are generally not involved in antigen recognition, but are involved in the activation of the complement system, and in the elimination of immune complexes (antibody bound to its antigen) by immune cells possessing constant fragment receptors (cFR). An antibody has four variable domains located at the ends of two “arms”. The association between a variable domain carried by a heavy chain (VH) and the adjacent variable domain carried by a light chain (VL) constitutes the antigen recognition site (or paratope). Thus, an immunoglobulin molecule has two antigen-binding sites, one at the end of each arm. These two sites are identical (but intended for different epitopes), making it possible to bind two antigen molecules per antibody. The antigen recognition site (or paratope) comprises 6 regions known as complementarity-determining regions (CDRs). Each VH has 3 CDRs, and each VL also has 3.

Specific enzymatic cleavage allows different fragments to be isolated:
the Fc fragment (fragment crystallizable). It is the basis of immunoglobulin's biological properties, in particular its ability to be recognized by immune effectors or to activate complement. It is made up of the constant fragments of the heavy chains (CH2) beyond the hinge region. It generally does not recognize the antigen;
the Fv fragment (Fragment variable). This is the smallest fragment of the immunoglobulin that retains the properties of the antibody. Consisting solely of the VL and VH variable regions, it binds the antigen with the same affinity as the full antibody, and is monovalent;
the Fab fragment (Fragment antigen-binding). This fragment has the same affinity for the antigen as the full antibody. The Fab fragment is made up of the entire light chain (VL+CL) and part of the heavy chain (VH+CH1). It is monovalent;
the F(ab′)2 fragment. It corresponds to the association of two Fab fragments linked by a small part of the constant parts of the heavy chains, the hinge region. It has the same affinity as the antibody for the antigen, and is divalent.

As used here, the term antibody encompasses native antibodies and their functional derivatives, (e.g. mutated and/or modified antibodies as well as mimetic antibodies), provided that such a derivative is capable of binding specifically to an antigen (referred to as “functional antibody derivatives”). As used herein, the term “antibody derivatives” also encompasses antibody fragments. Preferably, an “antibody fragment” is capable of binding specifically to an antigen (referred to as a “functional antibody fragment”).

Antibodies can be produced by various systems known to the skilled person. Antibody production systems include, for example, animal systems (such as rodents, camelids, etc.), hybridoma systems, mammalian cell systems (including, in particular, CHO cell lines (Chinese hamster ovary cells; e.g. CHO-K1, CHO-DG44, etc.), mouse myeloma cell lines (e.g. NSO), baby hamster kidney cell lines (e.g. BHK), human embryonic kidney cell lines (e.g. HEK293), etc.), yeast systems (including glycolization-enhanced yeast), insect cell systems (including glycolization-enhanced insect cell lines), plant cell systems (including glycolization-enhanced plant cell lines), etc. Preferably, the antibody is an animal antibody, preferably a mammalian antibody, more preferably a human or humanized antibody. Advantageously, the antibody is humanized.

The term “antibody” encompasses native antibodies and their derivatives (e.g. mutated and/or modified antibodies as well as mimetic antibodies), preferably provided that this derivative is capable of binding specifically to an antigen.

The various categories of antibodies and their production methods are well known to the skilled person, who will be able to refer in particular to reference works in the field (such as Thomas D. Pollard, William C. Earnshaw, Jennifer Lippincott-Schwartz, Graham Johnson Cell Biology E-Book, Elsevier Health Sciences, 1 Nov. 2016; Mohammed Zourob, Recognition Receptors in Biosensors, DOI 10.1007/978-1-4419-0919-0, Springer-Verlag New York 2010; Abbas, Lichtman, Pillai, Cellular and Molecular Immunology E-Book, Elsevier Health Sciences, Aug. 22, 2014; Bayer V., An overview of monoclonal antibodies. Semin Oncol Nurs. 2019 Sep. 2: 150927; Wang W, Wang E Q, Balthasar J P. Pharmacokinetics and pharmacodynamics of monoclonal antibodies. Clin Pharmacol Ther. 2008 November; 84(5):548-58).

The term “functional antibody fragment”, as used herein, refers to one or more part(s) or fragment(s) of an antibody, retaining the ability to bind specifically to an antigen. Examples of binding fragments encompassed by the term “functional antibody fragment” include, but are not limited to, an antigen-binding fragment (Fab), a Fab′ fragment, an F(ab′)2 fragment, a variable fragment (Fv), a single chain variable fragment (scFv), corresponding to the VH and VL regions fused by a linker peptide), a dsFv fragment (“disulfide-bond stabilised Fv”), a ds-scFv fragment (“disulfide-bond stabilised scFv”), a VH domain, a VL domain, a di-scFv (divalent scFv, consisting of the association of two scFvs), a diabody (consisting of the covalent or non-covalent association of two scFvs), a single-chain diabody, a triple body, a minibody (consisting of VL-VH-CH3 fragments), a nanobody, a single-domain antibody (sdAb), a single-chain antibody fragment (scAb), a heavy chain antibody (HcAb), a VHH, a variable new antigen receptor (VNAR), an immunoglobulin new antigen receptor (IgNAR), a bispecific T-cell engager (BITEs), a dual affinity retargeting molecule (DART), and any combination thereof (e.g., a fusion protein thereof).

The term “single-domain antibodies”, or “sdAb”, as used here, refers to antibody fragments consisting of a single monomeric variable domain of an antibody. These antibodies comprise only the monomeric variable regions of the heavy chain antibodies produced in particular by camelids or cartilaginous fish. Because of their different origins, they are also called VHH fragments (camelids) or VNAR (variable new antigen receptor; cartilaginous fish). Single-domain antibodies are also known as nanobodies. Single-domain antibodies can also be obtained by monomerization of the variable domains of conventional mouse or human antibodies through genetically engineering. They have a molecular mass of around 12-15 kDa and are therefore the smallest antibody fragments capable of recognizing an antigen.

The term “diabody” as used here refers to a fusion protein or bivalent antibody that can bind to different antigens. A diabody is composed of two unique protein chains that include fragments of an antibody, namely variable fragments. Diabodies comprise a variable heavy chain (VH) domain linked to a variable light chain (VL) domain on the same polypeptide chain (VH-VL, or VL-VH). Using a short peptide linking the two variable domains, the domains are forced to pair with the complementary domain of another chain, creating two antigen-binding sites. Diabodies can target the same antigen (monospecific) or different antigens (bispecific).

The terms “mimetic antibodies” or “antibody's mimetic” as used herein, refer to compounds that can bind specifically to antigens in an antibody-like manner, but are not structurally related to antibodies. Typically, antibody mimetics are peptides or engineered proteins with a molar mass of about 3 to 20 kDa that comprise one, two or more exposed antigen-specific binding domains. Examples of antibody mimetics include, but are not limited to, LACI-D1 (lipoprotein-associated coagulation inhibitor); affilins, e.g. human ubiquitin or human γ B crystallin; cystatin; Sac7D from Sulfolobus acidocaldarius; lipocalins and lipocalin-derived anticalins; DARPins (Designed Ankyrin Repeat Proteins); SH3 domain of Fyn; Kunits domains of protease inhibitors; monobodies, e.g. the 10th type III domain of fibronectin; adnectins; knottins (cysteine-knotted miniproteins); atrimers; evibodies; affibodies, e.g. the three-helix bundle of the Z domain of protein A from Staphylococcus aureus; Trans-bodies, e.g. human transferrin; tetranectins, e.g. the monomeric or trimeric domain of human C-type lectin; microbodies, e.g. trypsin-II inhibitor; armadillo repeat proteins; etc. Nucleic acids and small molecules can also be considered as antibody mimetics (e.g. aptamers), but not artificial antibodies, antibody fragments and fusion proteins composed from them. Common advantages over antibodies are better solubility, tissue penetration, heat and enzyme stability, and comparatively low production costs.

The terms “DARPin” or “Designed Ankyrin Repeat Protein” here refer to genetically engineered antibody mimetic proteins which generally exhibit highly specific, high-affinity binding to the target protein. They are derived from naturally occurring ankyrin repeat proteins, one of the most common classes of binding proteins in nature, which are responsible for diverse functions such as cell signaling, regulation and structural integrity. DARPins comprise, or consist essentially of, or consist of, at least three repeating motifs or modules, of which the most N- and C-terminal modules are called “caps”, as they protect the hydrophobic core of the protein. The number of internal modules is indicated by a number (e.g. N1C, N2C, N3C, . . . ) while the caps are indicated by “N” or “C”, respectively.

By “antigen” we mean a natural or synthetic molecule which, when recognized by antibodies or cells of an organism's immune system, is capable of triggering an immune response. In this way, any foreign substance or microbe introduced into the body can act as an antigen, triggering the production of special proteins-antibodies-which neutralize the harmful effects of the foreign substance. Antigens are generally peptides, proteins, sugars (such as polysaccharides or polyosides) and their lipid derivatives (lipids). Antigens can also be nucleic acids, or haptens (i.e. antigen fragments). Antigens, as markers of foreign agents, form the basis of the adaptive immune response. It is the recognition of the antigen by immunocompetent cells, either directly or via antigen-presenting cells (APCs), that activates specific immunity. In the case of protein antigens, the part of the antigen recognized by an antibody or lymphocyte receptor is called an “epitope” or “antigenic determinant”. The same antigen may comprise several epitopes (identical or different), thus inducing a varied immune response. There are sequential epitopes, corresponding to a sequence of amino acids, and conformational epitopes, linked to the structure of the protein and therefore sensitive to denaturation. Antigen recognition by lymphocytes depends on the nature of the epitope. B lymphocytes bind directly to conformational epitopes via their membrane immunoglobulins. T lymphocytes recognize sequential epitopes presented by antigen-presenting cells. The antigen may be exogenous, i.e. foreign to the individual (in this case, it may be allogeneic: derived from an individual of the same species; or xenogeneic: derived from other species), or it may be endogenous, i.e. an antigen specific to the host (autoantigens). The antigen is preferably a microorganism, plant, alga, microalgae, bacterial, viral, parasitic, yeast, fungal, insect, animal or tumor antigen; preferably a eukaryotic or prokaryotic pathogen or cancer antigen; preferably a bacterial, viral, parasitic, yeast, fungal or tumor protein, lipid or sugar, antigen.

The term antigen encompasses native antigens and their derivatives (e.g. mutated and/or modified antigens), preferably provided that this derivative is capable of being the target of an immune response.

The various categories of antigens are well known to the skilled person, who can refer in particular to reference works in the field (such as G. J. V. Nossal, G L Ada, Antigens, Lymphoid Cells and the Immune Response, Academic Press, 1971; Marc H. V. Van Regenmortel, Structure of Antigens, Volume 3 CRC Press, Dec. 20, 1995; Edouard Drouhet, Garry T. Cole, Louis De Repentigny, Jean Latge, Fungal Antigens: Isolation, Purification, and Detection, Springer Science & Business Media, Nov. 11, 2013; Graziano D. F., Finn O. J. (2005) Tumor Antigens and Tumor Antigen Discovery. In: Khleif S. N. (eds) Tumor Immunology and Cancer Vaccines. Cancer Treatment and Research, vol 123. Springer, Boston, MA; Wang M, Claesson M H, Methods Mol Biol. 2014; 1184:309-17. Classification of human leukocyte antigen (HLA) supertypes; as well as specialized databases as described in Galperin, Fernández-Suárez, Rigden, The 24th annual Nucleic Acids Research database issue: a look back and upcoming changes, NAR, Volume 45, Issue D1, January 2017, Pages D1-D11; in particular the PMAPP database, a database of human autoantigens (available notably at aagatlas.ncpsb.org)).

As used herein, an “antigen fragment” is any part of an antigen, preferably provided that this fragment/part is capable of being the target of an immune response (e.g. epitopes, immunogenic domains, etc.). In the case of a protein antigen, the antigenic fragment preferably comprises at least 6 consecutive amino acid residues of the antigen (preferably at least 8 consecutive amino acid residues of the antigen, preferably at least 10, preferably at least 15, preferably at least 20, preferably at least 30 amino acid residues of the antigen).

By “toxin” we mean a substance that is toxic to one or more living organisms. A toxin is typically synthesized by a living organism (bacteria, poisonous fungus, venomous insect or snake), to which it confers its pathogenic power. Toxins produced by bacteria are called bacteriotoxins, those produced by fungi are called mycotoxins, those produced by plants are called phytotoxins, those produced by algae are called phycotoxins, and those produced by animals are called animal toxins. The toxin may be a chemical molecule, a peptide, a protein, a glycoprotein, a sugar, an oside, a lipid, a nucleic acid, or any combination of these. Several families of bacteria secrete biotoxins (exotoxins) into the tissues they colonize. Other bacteria (Gram-negative) retain most of the toxic compounds within themselves, which are only released during cell lysis by chemical, physical or mechanical means (endotoxins). Toxic plants produce toxins via their secondary metabolites: these are molecules which, unlike primary toxins (proteins, lipids, carbohydrates, amino acids, etc.), are produced outside the metabolic pathways necessary for survival (i.e. primary metabolites). Plant toxins can be classified into three groups: phenols, nitrogen compounds and terpenes. The toxin may be a neurotoxin (a toxin acting on the nervous system), a myotoxin (acting on muscle contraction, notably cardiotoxins on the heart and others like strychnine on respiratory muscles), a hemotoxin (acting on the blood), a cytotoxin (acting on the cells), a dermatotoxin (acting on the skin and mucous membranes), a hepatotoxin (acting on the liver), a nephrotoxin (acting on the kidney), an enterotoxin (acting on the digestive tract) and so on. The toxin may be an anatoxin, i.e. a toxin which has been treated in such a way as to retain its antigenic power and lose its toxicity. The toxin is preferably a toxin of a microorganism, plant, alga, microalgae, bacterium, virus, parasite, yeast, fungus, insect, animal or tumor; preferably a toxin of a eukaryotic or prokaryotic pathogen, or of a cancer.

The various categories of toxins are well known to the skilled person, who will be able to refer in particular to reference works in the field (such as Michael W. Parker, Protein Toxin Structure, Springer Science & Business Media, Jun. 29, 2013; Michael R. Dobbs, Clinical Neurotoxicology E-Book: Syndromes, Substances, Environments, Elsevier Health Sciences, Jul. 22, 2009; Walker A A, Robinson S D, Yeates D K, Jin J, Baumann K, Dobson J, Fry B G, King G F. Entomo-venomics: The evolution, biology and biochemistry of insect venoms. Toxicon. 2018 November; 154:15-27; Vilariño N, Louzao M C, Abal P, Cagide E, Carrera C, Vieytes M R, Botana L M. Human Poisoning from Marine Toxins: Unknowns for Optimal Consumer Protection. Toxins (Basel). 2018 Aug. 9; 10(8); as well as specialized databases as described in Galperin, Fernandez-Suarez, Rigden, The 24th annual Nucleic Acids Research database issue: a look back and upcoming changes, NAR, Volume 45, Issue D1, January 2017, Pages D1-D11; in particular the Comparative Toxicogenomics Database, as described in Davis, Grondin Murphy, Johnson, Lay, Lennon-Hopkins, Saraceni-Richards, Sciaky, King, Rosenstein, Wiegers, Mattingly, The Comparative Toxicogenomics Database: update 2013, NAR, Volume 41, Issue D1, 1 Jan. 2013, Pages D1104-D1114 (available in particular at ctdbase.org)).

As used herein, a “toxin fragment” is any part of a toxin, preferably provided that such fragment/part is capable of being toxic to an organism and/or cell. In the case of a protein toxin, the toxin fragment preferably comprises at least 6 consecutive amino acid residues of the toxin (preferably at least 8 consecutive amino acid residues of the toxin, preferably at least 10, preferably at least 15, preferably at least 20, preferably at least 30 amino acid residues of the toxin).

The term “receptor” refers to a molecule in the cell membrane, cytoplasm or nucleus that binds specifically to a specific factor (a ligand, such as a neurotransmitter, hormone or other substance), inducing a cellular response to that ligand. Ligand-induced changes in receptor behavior lead to physiological modifications that constitute the ligand's “biological effects”. Receptors can comprise at least: a peptide, a protein, a glycoprotein, a sugar, an oside, a lipid, a nucleic acid, or any combination of these. Receptors are generally proteins or mixed proteins (modified proteins and/or associated with another molecule). The receptor may be a receptor on the outer part of the plasma membrane, a transmembrane receptor embedded in the lipid bilayer of cell membranes (usually a transmembrane protein, acting, for example, as a receptor for hormones and neurotransmitters—these receptors are either coupled to a G protein or carry an enzymatic or ion channel activity that enables activation of metabolic signal transduction pathways in response to ligand binding), or an intracellular receptor (these receptors can sometimes enter the cell nucleus to modulate the expression of specific genes, in response to activation by the ligand). The receptor is preferably a microorganism, plant, alga, microalga, bacterial, viral, parasitic, yeast, fungal, insect, animal or tumor receptor; preferably a eukaryotic or prokaryotic pathogen or cancer receptor; preferably a bacterial, viral, parasitic, yeast, fungal or tumor protein or glycoprotein receptor. The various categories of receptors are well known to the skilled person, who will be able to refer in particular to reference works in the field (such as Thomas D. Pollard, William C. Earnshaw, Jennifer Lippincott-Schwartz, Graham Johnson Cell Biology E-Book, Elsevier Health Sciences, Nov. 1, 2016; Mohammed Zourob, Recognition Receptors in Biosensors, DOI 10.1007/978-1-4419-0919-0, Springer-Verlag New York 2010; Abbas, Lichtman, Pillai, Cellular and Molecular Immunology E-Book, Elsevier Health Sciences, Aug. 22, 2014; as well as specialized databases as described in Galperin, Fernández-Suárez, Rigden, The 24th annual Nucleic Acids Research database issue: a look back and upcoming changes, NAR, Volume 45, Issue D1, January 2017, Pages D1-D11; in particular the GPCRdb database, as described in Isberg V., Mordalski S., Munk C., Rataj K., Harpsoe K., Hauser A. S., Vroling B., Bojarski A. J., Vriend G., Gloriam D. E., GPCRdb: an information system for G protein-coupled receptors. Nucleic Acids Res. 2016; 44: D356-D364 (available in particular at gpcrdb.org)).

As used herein, a “receptor fragment” is any portion of a receptor, preferably provided that this fragment/portion is capable of binding specifically to a specific factor (e.g. a ligand, hormone or other substance). In the case of a protein receptor, the receptor fragment preferably comprises at least 6 consecutive amino acid residues of the receptor (preferably at least 8 consecutive amino acid residues of the receptor, preferably at least 10, preferably at least 15, preferably at least 20, preferably at least 30 amino acid residues of the receptor).

By “enzyme” we mean a protein with catalytic properties. Virtually all biomolecules capable of catalyzing chemical reactions in cells are enzymes; some catalytic biomolecules, however, are made up of RNA and are therefore distinct from enzymes: these are ribozymes. An enzyme acts by lowering the activation energy of a chemical reaction, thereby increasing the reaction speed. The enzyme is not modified during the reaction. The initial molecules are the enzyme substrates, and the molecules formed from these substrates are the reaction products. Enzymes are characterized by their very high specificity. What's more, enzymes are reusable.

Enzymes are generally globular proteins that act alone or in complexes of several enzymes or subunits. Like all proteins, enzymes are made up of one or more polypeptide chains folded to form a three-dimensional structure corresponding to their native state.

Enzymes are molecules much larger than their substrates. Their size can vary from around 50 to over 2,000 residues. Only a very small part of the enzyme—usually between two and four residues, sometimes more—is directly involved in catalysis, the so-called catalytic site (or catalytic domain). The latter may be located close to one or more binding sites, at which the substrate(s) are bound and oriented to catalyze the chemical reaction. The catalytic site and the binding sites form the enzyme's active site.

Enzymes perform a wide range of functions in living organisms. For example, they can be involved in signal transduction and regulation of cellular processes, movement generation, active transmembrane transport, digestion, metabolism, the immune system, nucleic acid digestion, nucleic acid cleavage and nucleic acid production (referred to here as “nucleic acid enzymes”), and prodrug conversion (prodrug-to-drug conversion). The enzyme is preferably a prokaryotic, eukaryotic or viral enzyme, most preferably an enzyme from an animal, plant, alga, microalgae, insect, microorganism, bacterium, parasite, yeast, fungus or virus, and most preferably a mammalian enzyme, such as a human enzyme. The various categories of enzymes are well known to the skilled person, who will be able to refer in particular to reference works in the field (such as Schomburg D., Schomburg I., Springer Handbook of Enzymes. 2 edn. Heidelberg: Springer; 2001-2009; Liébecq C., IUPAC-IUBMB Joint Commission on Biochemical Nomenclature (JCBN) and Nomenclature Committee of IUBMB (NC-IUBMB) Biochem. Mol. Biol. Int. 1997; 43:1151-1156; IUBMB (1992), Enzyme Nomenclature 1992, Academic Press, San Diego; as well as specialized databases as described in Schomburg D, Schomburg I. Methods Mol Biol. 2010; 609:113-28. Enzyme databases; in particular the BRENDA database (available in particular at brenda-enzymes.org), as described for example by Chang A, Schomburg I, Placzek S, Jeske L, Ulbrich M, Xiao M, Sensen C W, Schomburg D, Nucleic Acids Res. 2015 January; 43. Epub 2014 Nov. 5. BRENDA in 2015: exciting developments in its 25th year of existence).

As used herein, an “enzyme fragment” is any portion of an enzyme, preferably provided that this fragment/portion is capable of enzymatic activity. In the case of a protein enzyme, the enzyme fragment preferably comprises at least 6 consecutive amino acid residues of the enzyme (and is preferably a catalytic site of the enzyme) (preferably at least 8 consecutive amino acid residues of the enzyme, preferably at least 10, preferably at least 15, preferably at least 20, preferably at least 30 amino acid residues of the enzyme).

By “Enzymatic activity” or “catalytic activity” or “activity” of an enzyme, we mean the efficiency of an enzyme in converting a substrate into a product in a given environment. Enzyme efficiency considers the rate at which the enzyme converts the substrate into a product, and the rate at which the enzyme converts the substrate into a product. By “rate of conversion of substrate to product by the enzyme” we mean the ratio between the quantity of final product obtained and the initial quantity of substrate for a defined quantity of enzyme. For example, enzyme activity in the sense of the invention can be expressed as the quantity of phloroglucinol produced in a given volume (in g/L).

By “hormone” we mean a biologically active chemical substance, generally synthesized by a glandular cell (usually following stimulation) and secreted into the internal environment in which it circulates (via the blood, lymph or sap). It transmits a message in chemical form (generally by acting on specific receptors on a target cell), and thus acts as a messenger in the body. It is capable of acting at very low doses.

The hormone is preferably a plant or animal hormone. Plant hormones are also known as phytohormones or growth factors. Their function is often to ensure plant growth or morphogenesis. Animal hormones are in most cases produced by the endocrine system (an endocrine gland or tissue).

Advantageously, the hormone is a vertebrate hormone, preferably selected from the following chemical classes:
Amino-derived hormones, which consist of a single amino acid (tyrosine or tryptophan) but in a derivative form.
Peptide hormones are chains of amino acids (proteins), the shorter ones being called peptides.
Steroid hormones, which are steroids derived from cholesterol.
Lipid- and phospholipid-based hormones.

The hormone is preferably selected from peptide or protein hormones, amine-derived hormones, steroid hormones and lipid hormones. The hormone is preferably an animal or plant hormone, preferably a mammalian hormone, most preferably a human hormone. The various categories of hormones are well known to the skilled person, who will be able to refer in particular to reference works in the field (such as Davies P. J. (2010) The Plant Hormones: Their Nature, Occurrence, and Functions. In: Davies P. J. (eds) Plant Hormones. Springer, Dordrecht; A W Norman, G Litwack, Hormones, Academic Press, 1997; A Kastin, Handbook of biologically active peptides, Academic Press, 2013).

As used herein, a “hormone fragment” is any portion of a hormone, preferably provided that this fragment/portion is capable of stimulating and/or inhibiting a biological process. In the case of a protein hormone, the hormone fragment preferably comprises at least 6 consecutive amino acid residues of the hormone (preferably at least 8 consecutive amino acid residues of the hormone, preferably at least 10, preferably at least 15, preferably at least 20, preferably at least 30 amino acid residues of the hormone).

The term “ligand” generally refers to a substance that binds to a cell receptor and induces a biological signal. In particular, the term ligand encompasses the terms “addressing or targeting or transport signal”, “signaling molecule”, “signal”, and “cellular signal”. Examples of ligands include peptide and protein addressing sequences, oligosaccharides, molecules enabling cellular transport and/or internalization, neurotransmitters, receptor ligands (receptors being as defined above), as well as cellular recognition molecules such as Toll Like receptor ligands or C-type lectin receptor ligands. An addressing sequence is a short amino acid sequence, generally located at the N-terminus of the protein, used to designate the proteins to be addressed, and to indicate their destination. Thus, the addressing or targeting or transport signal can be an addressing or targeting or transport signal to/from the nucleus; an addressing or targeting or transport signal to/from the cytoplasm; an addressing or targeting or transport signal to/from the cytosol; an addressing or targeting or transport signal to/from the cell membrane; an addressing or targeting or transport signal to/from mitochondria; an addressing or targeting or transport signal to/from peroxisomes; an addressing or targeting or transport signal to/from lysosomes; an addressing or targeting or transport signal to/from the endoplasmic reticulum; an addressing or targeting or transport signal to/from secretory pathways; and a ligand for a receptor, preferably a membrane or transmembrane receptor, preferably a membrane or transmembrane receptor for a membrane selected from a cell membrane, an extracellular membrane, a cytoplasmic membrane or a nuclear membrane. The addressing or targeting or transport signal may comprise at least: a peptide, a protein, a glycoprotein, a sugar, an oside, a lipid, a nucleic acid, or any combination thereof. Preferably, the addressing or targeting or transport signal comprises at least one peptide, protein, glycoprotein, or nucleic acid. The signal is preferably a prokaryotic, eukaryotic or viral signal, most preferably a signal from an animal, a plant, an alga, a microalgae, a microorganism, a bacterium, a parasite, a yeast, a fungus, an insect, a virus, or a cancer; even more preferably a mammalian signal, such as a human signal. The various categories of signals are well known to the skilled person, who will be able to refer in particular to reference works in the field (such as Thomas D. Pollard, William C. Earnshaw, Jennifer Lippincott-Schwartz, Graham Johnson Cell Biology E-Book, Elsevier Health Sciences, 1 Nov. 2016; Mohammed Zourob, Recognition Receptors in Biosensors, DOI 10.1007/978-1-4419-0919-0, Springer-Verlag New York 2010; Abbas, Lichtman, Pillai, Cellular and Molecular Immunology E-Book, Elsevier Health Sciences, Aug. 22, 2014).

A “ligand fragment” is any portion of a ligand, preferably provided that this fragment/portion is capable of binding to a cell receptor and inducing a biological signal. In the case of a protein ligand, the ligand fragment preferably comprises at least 6 consecutive amino acid residues of the ligand (preferably at least 8 consecutive amino acid residues of the ligand, preferably at least 10, preferably at least 15, preferably at least 20, preferably at least 30 amino acid residues of the ligand).

By “nanoparticle” we mean an object whose three dimensions are on the nanometric scale, i.e. a particle with a nominal diameter of less than approximately 100 nm (for example, as defined by ISO standard TS/27687).

By “Functional group” or “functional group” we mean a reactive group, i.e. one with the ability to form at least one chemical, biological, biochemical or enzymatic reaction, or any combination thereof, with another molecule. By “agent-binding functional group” we mean a functional group possessing the ability to form at least one chemical, biological, biochemical or enzymatic reaction, or any combination thereof, with an agent (notably chosen from a therapeutic agent, a targeting agent, a marking agent, and any combination thereof).

The functional group may for example comprise, or consist essentially of, or consist of, a peptide tag, a chemical group (such as a clickable function, a crosslinking group, and any combination thereof), an antibody, an antibody derivative, a functional fragment of an antibody or its derivative, an affinity tag (e.g. biotin, streptavidin, chitin-binding protein (CBP), maltose-binding protein (MBP), Strep-tag, glutathione-S-transferase (GST), poly(His) tag, etc.), or any combination thereof.

By “clickable function”, or “chemistry-click” or “fast bio-orthogonal chemistry”, we mean a chemical group capable of reacting with another chemical group, in the absence of solvent, at physiological pH, without the formation of residue or by-product. Examples of clickable functions include, but are not limited to, azide groups, alkyne groups (e.g. acetylene), and any combination thereof. In particular, the clickable function may be selected from an N-hydroxysuccinimide (NHS), dibenzocyclooctyne (DBCO), tetrazine, methyl-tetrazine group, and any combination thereof.

As used herein, the terms “peptide label”, or “peptide tag”, refer to a peptide sequence between 6 and 400 amino acids (preferably between 8 and 300 amino acids, more preferably between 10 and 200 amino acids, more preferably between 12 and 82 amino acids). Examples of peptide labels include affinity peptide labels, solubilization peptide labels, chromatography peptide labels, epitope peptide labels, fluorescence peptide labels and so on. Affinity peptide tags are generally added to proteins so that they can be purified from their crude biological source using an affinity technique. Examples include chitin-binding protein (CBP), maltose-binding protein (MBP), Strep-tag, glutathione-S-transferase (GST), poly(His) tag, etc. Solubilizing peptide tags are particularly used for proteins expressed in chaperone-deficient species, such as E. coli, to aid correct protein folding and prevent precipitation. These include thioredoxin (TRX) and poly(NANP). Some affinity peptide labels have a dual role as solubilizing agents, such as MBP and GST. Chromatographic tags are used to modify the chromatographic properties of the protein to enable different resolution in a particular separation technique. They often consist of polyanionic amino acids, such as the FLAG-tag. Epitope tags are short peptide sequences chosen because high-affinity antibodies can be reliably produced in many different species. They are generally derived from viral genes. Epitope tags include the ALFA tag, the V5 tag, the Myc tag, the HA tag, the Spot tag, the T7 tag, the NE tag and others. Fluorescence tags are used in particular to give a visual reading of a protein. GFP and its variants are the most commonly used fluorescence tags. Peptide tags can enable specific enzymatic modification (such as biotinylation by biotin ligase) or chemical modification (such as reaction with FlASH-EDT2 for fluorescence imaging). Peptide tags can be combined, in particular to link proteins to several other components. Peptide tags also include covalent peptide tags. Examples of covalent peptide tags include, but are not limited to:
Isopeptag (covalently binding to the pilin-C protein);
SpyTag (covalently binding to the SpyCatcher protein);
SnoopTag (covalently binding to the SnoopCatcher protein);
SnoopTagJr (covalently binding to the SnoopCatcher protein or the DogTag protein (mediated by SnoopLigase));
DogTag (covalently binding to the SnoopTagJr protein, mediated by SnoopLigase),
SdyTag (covalently binding to the SdyCatcher protein);
Every variation thereof.

Label peptide variants are well described in the literature, are available to the skilled person and need not be described in detail herein.

By “Illness” or “disease” or “disorder” or “pathology” (these terms are considered synonymous here), we mean an alteration in the functions or health of a living organism. This includes both disease, which refers to all alterations in health, and illness, which designates a particular entity characterized by its own causes, symptoms, course and therapeutic possibilities.

By “Prevention” or “prevention of a disease” or “prevention of the onset of a disease” we mean reducing the risk of the onset, development or amplification of a disease, the causes of a disease, the symptoms of a disease, the effects (or consequences, preferably the deleterious effects/consequences) of a disease, or any combination thereof; and/or delaying the onset, development or amplification of a disease, the causes of a disease, the symptoms of a disease, the effects (or consequences, preferably the harmful, deleterious effects/consequences) of a disease, or any combination thereof. Prevention includes, in particular, preventive treatments.

By “treatment” or “treatment of a disease” we mean the reduction, inhibition and/or disappearance of a disease, the causes of a disease, the symptoms of a disease, the effects (or consequences, preferably the harmful, deleterious effects/consequences) of a disease, or any combination of these. Treatment is preferably curative.

A “treatment” or “therapy” includes, but is not limited to, one or more molecules and/or drugs (including any type of molecule or drug, such as chemical or biological compounds, antibodies, antigens, gene therapy, cell therapy, immunotherapy, chemotherapy, any combination thereof, etc.), and/or other treatments (such as radiotherapy, immunotherapy, chemotherapy, surgery, endoscopy, interventional radiology, physical oncology, phototherapy, etc.).), and/or other treatments (such as radiotherapy, immunotherapy, chemotherapy, surgery, endoscopy, interventional radiology, physical oncology, phototherapy, light therapy, ultrasound therapy, thermotherapy, cryotherapy, electrotherapy, electroconvulsive therapy, oxygen therapy, assisted ventilation, hydrotherapeutic massage, organ/tissue/fluid transplantation, implantation, and any combination thereof, etc.).) A therapy can be delivered by different modes of administration. The skilled person know how to select the most appropriate mode(s) of administration, depending on the therapy, the disease and the subject to be treated. For example, modes of administration include, but are not limited to, oral administration, administration by injection into a vein (intravenous, IV), into a muscle (intramuscular, IM), into the space around the spinal cord (intrathecal), under the skin (subcutaneous, sc); sublingual administration; buccal administration; rectal administration; vaginal administration; ocular route; otic route; nasal administration; by inhalation; by nebulization; cutaneous, topical or systemic administration; transdermal administration.

“Medicament” or “drug” means any substance or composition represented as having curative or preventive properties with respect to human or animal diseases. A medicament therefore includes any substance or composition that can be used in or administered to humans or animals for the purpose of making a medical diagnosis or restoring, correcting or modifying their physiological functions by exerting a pharmacological, immunological or metabolic action. The term medicament notably includes vaccines.

The terms “therapeutic” or “therapeutic uses” in the context of the present invention cover “prevention”, “treatment” and “vaccine” uses.

A “therapeutically effective quantity” is the amount of each active ingredient that is sufficient to produce a beneficial health outcome. An “immunologically effective quantity” corresponds to the quantity of each active entity that is sufficient to produce a detectable immune response. The active entity is, for example, an active ingredient, a therapeutic agent, a targeting agent, a labeling agent, or any combination of these.

As used herein, a “pharmaceutically acceptable excipient” or “pharmaceutically acceptable carrier” is intended to include all carriers, solvents, diluents, excipients, adjuvants, vehicles, dispersion media, coatings, antibacterial and antifungal agents, absorption-retarding agents, and the like, compatible with administration in any subject including animals, and in particular humans. Suitable carriers for use herein are well known in the art (see, for example, the most recent edition of Remington: The Science and Practice of Pharmacy, A. Gennaro, Lippincott, Williams&Wilkins). Non-limiting examples of excipients include water, NaCl, saline solutions, saccharide solutions (e.g. glucose, trehalose, sucrose, dextrose, etc.), lactated Ringer's, alcohols, oils, gelatins, carbohydrates such as lactose, amylose or starch, fatty acid esters, hydroxymethylcellulose, etc. In particular, swelling agents such as a sugar like lactose, sucrose, trehalose, sorbitol, glucose, raffinose or mannitol, preferably lactose, can be used as adjuvants, sucrose, trehalose, glucose or mannitol, an amino acid such as arginine, glycine or histidine, preferably glycine, or polymers of the dextran or polyethylene glycol type, or mixtures thereof.

By “subject” or “patient” we mean a human individual or an animal other than a human. The subject is, for example, a human or animal likely to contract a disease, likely to be affected by a disease, or suffering from a disease. The subject is preferably a human being. The subject may be a child (human subject aged 16 or under) or an adult (human subject aged over 16). By “healthy subject” we mean a subject who does not suffer from the disease in question. In the context of the present invention, a healthy subject is preferably one who does not suffer from any disease. By “Reference subject” we mean a subject suffering from a known disease at a known stage.

By “biological sample” or “specimen” from a subject, we mean a whole organ or tissue or part of such organ or tissue, a fluid or fraction of such fluid, cells or cellular components, obtained from such subject, as well as a homogenate, lysate or extract prepared therefrom. In particular, a “biological sample” or “specimen” is preferably any tissue (preferably portions or fractions thereof) that can be used to detect disease, including, but not limited to, plasma, blood, lymph, serum, urine, mucus, saliva, a central nervous system (CNS; such as a brain sample or spinal cord sample, etc.), a respiratory tract sample (such as a lung sample, etc.), a salivary gland sample, a nasopharyngeal sample, an oropharyngeal sample, a digestive system sample (e.g. colon, intestine, etc.), a skin sample, an organ sample (e.g. liver, kidney, spleen, etc.), etc.

The biological sample may have been previously obtained by any technique known in the art. Such techniques include, for example, swabbing, needle or syringe sampling, surgery (such as stereotactic surgery), puncture, explant, excision, biopsy. By “excision” we mean a surgical procedure consisting in cutting (excising) a more or less wide or deep part of the tissue, preferably a tissue abnormality or growth. An excision may be performed to remove and/or analyze a cancerous or suspicious tumor. The term “biopsy” here refers to a sample of cells or tissue taken for analysis. Several types of biopsy procedures are known and practiced in the field. The most common types include (1) incisional biopsy, in which only a sample of tissue is removed; (2) excisional biopsy (or surgical biopsy), in which a tumor mass is completely removed, thus performing a therapeutic and diagnostic procedure; and (3) needle biopsy, in which a sample of tissue is removed using a needle, which may be large or fine. Other types of biopsies exist, such as smears or curettage, and can also be used to obtain the sample. Consequently, the sample can be, for example, an explant, an excision, a biopsy, etc. The sample is preferably obtained by a minimally invasive procedure, such as stereotactic surgery.

In the detailed description which follows, the embodiments may be taken alone or in any combination suitable by the skilled person, and the above definitions apply to all the embodiments described below as well as to combinations thereof.

### Lipid Microbubble

In the context of the present invention, the Inventors have developed innovative microbubbles capable of delivering agents of interest into the body in a precise, targeted manner.

In particular, the Inventors have shown that, surprisingly, the lipid microbubbles developed this way possess significantly improved stability, unlike the microbubbles described in the prior art. Remarkably, the data also reveal that these optimized microbubbles are capable of delivering different types of therapeutic agents, targeting agents and/or labeling agents, including nucleic acids, more effectively than microbubbles described in the prior art. In particular, these microbubbles are capable of actively crossing vessels, the blood-brain barrier (BBB), or the tumor microenvironment. The Inventors have also demonstrated that the localized application of ultrasound can target these optimized microbubbles very precisely to the area to be treated. These data thus reveal the therapeutic potential of these lipid microbubbles to treat a wide range of pathologies in a targeted manner, including central nervous system pathologies, vessel pathologies, tumors and cancers.

The data also show that these optimized microbubbles can be used as marking, detection and imaging tools.

This high degree of flexibility is due in particular to the original formulation of the microbubbles, which uses a mixture of cationic molecules such as lipophosphoramidates and/or histidylated polyethyleneimine.

The present invention therefore concerns a lipid microbubble, comprising, or consisting essentially of, or consisting of, at least one cationic compound selected from lipophosphoramidates, histidylated polyethylenimines, and any mixture thereof. Advantageously, the envelope of the lipid microbubble comprises, or consists essentially of, or consists of, at least one cationic compound chosen from lipophosphoramidates, histidylated polyethylenimines, and any mixture thereof. Thus, according to an advantageous embodiment, the present invention relates to a lipid microbubble having an envelope comprising, or consisting essentially of, or consisting of, at least one cationic compound selected from lipophosphoramidates, histidylated polyethylenimines, and any mixture thereof.

The lipid microbubble according to the invention can in particular be an ultrasound contrast agent.

According to a preferred embodiment, the microbubble according to the invention further comprises at least one agent selected from a therapeutic/pharmacological agent, a targeting agent, a marking agent, and any combination thereof.

The therapeutic/pharmacological agent, targeting agent, labeling agent, and their combination is (are) preferably:
i. exposed to the surface of the microbubble, or
ii. embedded in the microbubble's lipid envelope, or
iii. incorporated inside the microbubble, or
iv. any combination of i to iii.

The therapeutic/pharmacological agent, targeting agent and labeling agent are advantageously as defined in the “Definitions” section above.

According to one embodiment, said at least one agent comprises, or consists essentially of, or consists of, or is/are selected from:
1) a nucleic acid;
2) a liposoluble active ingredient;
3) a chemotherapeutic agent, such as a cytotoxic agent and/or a cytostatic agent;
4) an antibody;
5) a protein;
6) an antigen;
7) a toxin;
8) a receiver
9) an enzyme;
10) a hormone;
11) a ligand;
12) a viral vector;
13) a nanoparticle, preferably comprising at least one agent selected from a therapeutic agent, a targeting agent, a marking agent, and any combination thereof;
14) any derivative of 1) to 13), preferably any functional derivative thereof;
15) any fragment from 1) to 14), preferably any functional fragment thereof; and
16) any combination of 1) to 15).

According to a particularly preferred embodiment, said at least one agent comprises, or consists essentially of, or consists of, or is/are selected from:
a) a nucleic acid;
b) a liposoluble active ingredient;
c) a chemotherapeutic agent, such as a cytotoxic agent and/or a cytostatic agent;
d) an antibody;
e) an antibody derivative;
f) a functional fragment of an antibody or antibody derivative;
g) a protein;
h) a protein fragment, such as a peptide (e.g. a cell-penetrating peptide (CPP), an antigen, an epitope, a functional protein domain, etc.);
i) a nanoparticle, preferably comprising (which may further comprise) at least one agent selected from a therapeutic agent, a targeting agent, a marking agent, and any combination thereof (including a hydrophilic/non-liposoluble agent); and
j) any combination of a) to i).

The various types of agents 1) to 16), and a) to i), as listed above, are advantageously as defined in the “Definitions” section above.

Preferably, nucleic acid 1) and/or a) comprises, consists essentially of, or consists of, a plasmid, a vector, complementary DNA (cDNA), single-stranded DNA, double-stranded DNA, DNA comprising a sequence encoding a gene or gene fragment, DNA encoding a gene or gene fragment (preferably a functional fragment), RNA, double-stranded RNA, messenger RNA, non-coding RNA, small RNA and so on.

Preferably, chemotherapeutic agent 3) or c) is selected from cytotoxic agents, cytostatic agents, and cytotoxic and cytostatic agents. In particular, the chemotherapeutic agent can be chosen from, but is not limited to, paclitaxel, doxorubicin, gencitabin (e.g. Gemzar), temozolomide, etc.

The targeting agent may in particular comprise, consist essentially of, or consist of, an antibody, an antibody derivative, a functional fragment of an antibody or its derivative, a protein, a protein fragment (such as a peptide, an antigen, an epitope, a protein functional domain, etc.), a nanoparticle (which may further comprise at least one therapeutic agent, in particular a hydrophilic therapeutic agent), and any combination thereof.

According to an advantageous embodiment, the microbubble according to the invention further comprises at least one functional group, in particular a functional group enabling binding to at least one agent selected from a therapeutic agent, a targeting agent, a marking agent, and any combination thereof. Said group is preferably:
i. exposed to the surface of the microbubble, or
ii. embedded in the microbubble's lipid envelope, or
iii. incorporated inside the microbubble, or
iv. any combination of i to iii.

The functional group is advantageously as defined in the “Definitions” section above. Advantageously, the functional group comprises, or consists essentially of, or consists of, or is selected from:
a) a peptide label;
b) a chemical group, preferably selected from a clickable function, a coupling group, and any combination thereof;
c) an antibody;
d) an antibody derivative;
e) a functional fragment of an antibody or antibody derivative;
f) an affinity tag (e.g. biotin, streptavidin, chitin-binding protein (CBP), maltose-binding protein (MBP), Strep-tag, glutathione-S-transferase (GST), poly(His) tag, etc.); and
g) any combination of a) to f).

According to one embodiment, the lipophosphoramidate comprises, or consists essentially of, or consists of, or is selected from the group consisting of, a dimyristoyl phosphoramidate (preferably selected from a dimyristoyl bromide phosphoramidate (preferably O,O-dimyristoyl-N-[3N—(N methylimidazolium bromide) propylene] phosphoramidate (compound KLN27)), a dimyristoyl histamine phosphoramidate (preferably O,O-dimyristoyl(-N-(histamine)phosphoramidate (compound MM30)), and any combination thereof), a dioleyl phosphoramidate (preferably selected from the group of dioleyl methylimidazolium phosphoramidates (preferably O,O-dioleyl-N-(3 N—(N-methylimidazolium iodide) propylene) phosphoramidate (compound KLN25)), a dipalmitoyl phosphoramidate, a disteraoyl phosphoramidate, and any mixture thereof.

In one embodiment, the histidylated polyethylenimine is coupled to a fatty acid, preferably chosen from stearic acid, myristic acid, palmitic acid, oleic acid and any combination thereof.

Advantageously, the microbubble further comprises (in particular the microbubble envelope further comprises) an additional lipid selected from dimyristoyl-glycero-phosphocholine (preferably 1,2-dimyristoyl-sn-glycero-3-phosphocholine (DMPC)), distearoyl-glycero-phosphocholine (preferably 1,2-distearoyl-sn-glycero-3-phosphocholine (DSPC)), dimyristoyl-glycero-phosphoethanolamine-(polyethylene glycol) (preferably 1,2-dimyristoyl-sn-glycero-3-phosphoethanolamine-N-[methoxy (polyethylene glycol)-2000] (DMPE-PEG2000)), distearoyl-glycero-phosphoethanolamine-(polyethylene glycol) (preferably 1,2-distearoyl-sn-glycero-3-phosphoethanolamine-N-[amino(polyethylene glycol)-2000] (DSPE-PEG2000)), and a distearoyl-glycero-phosphoethanolamine-[biotinyl(polyethylene glycol)] (preferably 1,2-distearoyl-sn-glycero-3-phosphoethanolamine-N-[biotinyl (polyethylene glycol)-2000] (DSPE-PEG2000-biotin)), cholesterol, beta-sitosterol, 1,2-dioleoyl-sn-glycero-3-phosphoethanolamine (DOPE), 1-oleoyl-2-[6-[(7-nitro-2-1,3-benzoxadiazol-4-yl)amino]hexanoyl]-3-trimethylammonium propane (DOTAP), a 1,2-dimyristoyl-rac-glycero-3-methoxypolyethylene glycol-2000 (DMG-PEG2000), and any combination thereof.

In a preferred embodiment, the microbubble also contains a biocompatible gas. The biocompatible gas is preferably contained by the microbubble envelope (i.e. it is inside the microbubble, in the medium/cavity formed by the envelope). The biocompatible gas is preferably selected from perfluorobutane (C4F10), perfluoropropane (C3F8), dinitrogen (N2), sulfur hexafluoride (SF6), nitrogen oxide (NO), hydrogen, dioxygen, helium, xenon, argon, nitrous oxide (N2O), and any mixture thereof; preferably selected from perfluorobutane (C4F10), perfluoropropane (C3F8), dinitrogen (N2), sulfur hexafluoride (SF6), oxygen, nitrous oxide (N2O), and mixtures thereof. Particularly preferred, the gas is selected from the perfluorobutane group (C4F10).

Data obtained by the Inventors reveal that the microbubbles according to the invention, as defined above, are remarkably capable of actively crossing vessels, the blood-brain barrier (BBB) and/or the tumor microenvironment (particularly during localized application of ultrasound). The Inventors have also demonstrated that the localized application of ultrasound enables these microbubbles to be targeted very precisely to the area to be treated, including difficult-to-access areas such as the central nervous system, vessels, and the tumor microenvironment. Furthermore, the data also show that these optimized microbubbles are capable of efficiently delivering different types of therapeutic agents, targeting agents and/or labeling agents, including nucleic acids, to these hard-to-reach areas.

Thus, according to an advantageous embodiment, the microbubble according to the invention is characterized in that it is capable of actively crossing the blood-brain barrier and/or the tumor microenvironment (in particular during the localized application of ultrasound).

### Methods for Producing a Lipid Microbubble

The present invention also relates to a method of producing at least one microbubble according to the invention, as described above, comprising, or consisting essentially of, or consisting of, the following steps:
a) A mixture, in a container, of cationic compounds selected from lipophosphoramidates, histidylated polyethylenimines, and any mixtures thereof; ethanol; and optionally at least one agent selected from a therapeutic agent, a targeting agent, a marking agent, and any combination thereof;
b) Evaporation of the mixture obtained in step a) to obtain a lipid film and rehydration of the lipid film to form a liposomal suspension; this entire step can also be performed using microfluidics;
c) Lyophilization of the liposomal suspension obtained in step b);
d) Replacement of the air contained in the container containing the lyophilizate obtained in step c), by a biocompatible gas, the gas preferably being selected from perfluorobutane (C4F10), perfluoropropane (C3F8), dinitrogen (N2), sulfur hexafluoride (SF6), nitrogen oxide (NO), hydrogen, dioxygen, helium, xenon, argon, nitrous oxide (N2O), and any mixture thereof;
e) Rehydration of the lyophilizate from step d) to obtain a solution;
f) Stirring the solution obtained in step d) to form microbubbles;
g) Optionally, functionalization of the microbubbles with at least one agent selected from a therapeutic agent, a targeting agent, a marking agent, and any combination thereof; and/or functionalization of the microbubbles with the addition of at least one functional group enabling binding to at least one agent selected from a therapeutic agent, a targeting agent, a marking agent, and any combination thereof.

The microbubble and its constituents, such as lipophosphoramidate, histidylated polyethylenimine, therapeutic agent, targeting agent, marking agent, biocompatible gas, are advantageously as described in the preceding sections (in the “Definition” and/or “Lipid microbubble” section).

The invention also relates to a microbubble obtainable, or obtained, or directly obtained, by the production method described above.

### Compositions and Kits

The present invention further relates to a kit comprising, or consisting essentially of, at least one microbubble according to the invention, as defined above.

The present invention also relates to a composition comprising, or consisting essentially of, or consisting of, at least one microbubble according to the invention, as defined above, and, optionally, an excipient.

The present invention relates in particular to a pharmaceutical composition comprising, or consisting essentially of, or consisting of, at least one microbubble according to the invention, as defined above, and, optionally, a pharmaceutically acceptable excipient.

Advantageously, the composition, in particular the pharmaceutical composition, comprises a therapeutically effective amount of microbubbles. The concentration of microbubbles in the composition, in particular the pharmaceutical composition, preferably ranges from 10⁶ to 10¹⁴ microbubbles/ml, even more preferably from 10⁷ to 10¹³ microbubbles/ml, even more preferably from 10⁸ to 10¹² microbubbles/ml, even more preferably from 10⁹ to 10¹¹ microbubbles/ml, even more preferably the concentration of microbubbles in the composition being about 10¹⁰ microbubbles/ml.

According to one embodiment, the composition, in particular the pharmaceutical composition, comprises an amount of pharmaceutically acceptable excipient in the composition ranging from 5% to 99% by weight based on the total weight of the composition, preferably from 10 to 97% by weight, preferably from 20 to 95% by weight, preferably from 30 to 90% by weight, preferably from 40 to 85% by weight, preferably from 50 to 80% by weight, preferably from 60 to 70% by weight, based on the total weight of the composition.

According to one embodiment, the kit, comprises, or consists essentially of, or consists of:
a) at least one microbubble according to the invention (as defined above) or a composition as defined above, in particular a pharmaceutical composition as defined above, in a first container;
b) at least one therapeutic agent, in a second container;
c) optionally, at least one targeting agent in a third container;
d) optionally, at least one marking agent in a fourth container; and
e) optionally, instructions for preparation and/or use.

Advantageously, the kit also comprises means suitable for detecting the presence or absence of the marking agent in a sample and/or subject.

The microbubble and its constituents, such as lipophosphoramidate, histidylated polyethylenimine, therapeutic agent, targeting agent, marking agent, biocompatible gas, are advantageously as described in the preceding sections (in the “Definition”, and/or “Lipid microbubble”, and/or “Methods of producing a lipid microbubble” section).

Thus, preferably, the therapeutic agent and/or targeting agent and/or labeling agent comprises, consists essentially of, or consists of, or is selected from:
1) a nucleic acid;
2) a liposoluble active ingredient;
3) a chemotherapeutic agent, such as a cytotoxic agent and/or a cytostatic agent;
4) an antibody;
5) a protein;
6) an antigen;
7) a toxin;
8) a receiver
9) an enzyme;
10) a hormone;
11) a ligand;
12) A viral vector;
13) a nanoparticle, preferably comprising at least one agent selected from a therapeutic agent, a targeting agent, a marking agent, and any combination thereof;
14) any derivative of 1) to 13), preferably any functional derivative thereof;
15) any fragment from 1) to 14), preferably any functional fragment thereof; and
16) any combination of 1) to 15);

More preferably from a nucleic acid, a liposoluble active ingredient, a chemotherapeutic agent, an antibody, an antibody derivative, a functional fragment of an antibody or its derivative, a protein, a protein fragment, a nanoparticle, and any combination thereof.

### Therapeutic Uses and Methods

The Inventors have notably shown that, surprisingly, the innovative microbubbles developed here possess the ability to deliver agents of interest into the body in a precise and targeted manner, including into very difficult-to-access areas such as the central nervous system, vessels and the tumor microenvironment. Indeed, the Inventors have notably shown that, surprisingly, lipid microbubbles are significantly more stable than prior art microbubbles. In particular, they are capable of actively crossing vessels, the blood-brain barrier (BBB) and the tumor microenvironment. The Inventors have also demonstrated that the localized application of ultrasound enables these optimized microbubbles to be targeted very precisely to the area to be treated. These data thus reveal the therapeutic potential of these lipid microbubbles to treat a wide range of pathologies in a targeted manner, including pathologies of the central nervous system.

The data also show that these optimized microbubbles can be used as marking, detection and imaging tools.

The present invention therefore provides both powerful, broad-spectrum treatment methods for pathologies, as well as marking methods, particularly for medical imaging.

The present invention therefore relates to a microbubble according to the invention (as defined above); or a pharmaceutical composition or kit comprising, or consisting essentially of, at least one microbubble according to the invention (as defined above); or a pharmaceutical composition as defined above; or a kit as defined above; or any combination thereof; for use as a medicament. The present invention also relates to a microbubble according to the invention (as defined above); or a pharmaceutical composition or kit comprising, or consisting essentially of, at least one microbubble according to the invention (as defined above); or a pharmaceutical composition as defined above; or a kit as defined above; or any combination thereof; for use as a marking agent, in particular as a contrast agent.

The present invention also relates to the use of a microbubble according to the invention (as defined above); or a pharmaceutical composition or kit comprising, or consisting essentially of, at least one microbubble according to the invention (as defined above); or a pharmaceutical composition as defined above; or a kit as defined above; or any combination thereof; as a medicament.

The present invention also relates to the use of a microbubble according to the invention (as defined above); or a pharmaceutical composition or kit comprising, or consisting essentially of, at least one microbubble according to the invention (as defined above); or a pharmaceutical composition as defined above; or a kit as defined above; or any combination thereof; as a marking agent, in particular as a contrast agent.

The present invention also relates to the use of a microbubble according to the invention (as defined above); or a pharmaceutical composition or kit comprising, or consisting essentially of, at least one microbubble according to the invention (as defined above); or a pharmaceutical composition as defined above; or a kit as defined above; or any combination thereof; for the manufacture of a medicament.

The present invention also relates to the use of a microbubble according to the invention (as defined above); or a pharmaceutical composition or kit comprising, or consisting essentially of, at least one microbubble according to the invention (as defined above); or a pharmaceutical composition as defined above; or a kit as defined above; or any combination thereof; for the manufacture of a marking agent, in particular a contrast agent.

The present invention also relates to a method of treatment, comprising administering a microbubble according to the invention (as defined above); or a pharmaceutical composition or kit comprising, or consisting essentially of, at least one microbubble according to the invention (as defined above); or a pharmaceutical composition as defined above; or a kit as defined above; or any combination thereof; to a subject (preferably a subject in need thereof).

The present invention also relates to a marking method, comprising administering a microbubble according to the invention (as defined above); or a pharmaceutical composition or kit comprising, or consisting essentially of, at least one microbubble according to the invention (as defined above); or a pharmaceutical composition as defined above; or a kit as defined above; or any combination thereof; to a subject (preferably a subject in need thereof).

The microbubble and its constituents, such as lipophosphoramidate, histidylated polyethylenimine, therapeutic agent, targeting agent, marking agent, biocompatible gas, are advantageously as described in the preceding sections (in the “Definition”, and/or “Lipid microbubble”, and/or “Methods of producing a lipid microbubble” section).

Advantageously, the pharmaceutical composition and/or kit are as described above in the “Compositions and kits” section.

According to a preferred embodiment, the microbubble, composition, kit, or any combination thereof, is/are administered to a subject in need thereof, preferably in a therapeutically effective amount.

The pathology to be treated by administration of the microbubble, the composition, the kit, or any combination thereof (in the context of the aforementioned uses and treatment methods) can be of any type. In particular, the pathology may be selected from vessel pathologies, central nervous system pathologies, tumors, cancers, and any combination thereof.

The area to be marked by the administration of the microbubble, the composition, the kit, or any combination thereof (in the context of the aforementioned uses and treatment methods) may be of any type. The area to be marked can be any part of the body of the subject to be treated and/or marked. In particular, the area to be marked can be chosen from a vessel area, a central nervous system area, a tumor microenvironment area, and any combination thereof.

Thus, the area to be treated and/or the area to be marked can advantageously be located in the vessels, the central nervous system, the tumor microenvironment, or any combination thereof (preferably the central nervous system).

The microbubble, composition, kit, or any combination thereof, is (are) preferably formulated to be administered one or more times by the same or different routes. All conventional routes of administration are applicable in the context of the invention, including oral, parenteral and topical. Parenteral routes are intended for administration by injection or infusion, and include systemic as well as local routes. Preferably, the microbubble, composition, kit, or any combination thereof, is formulated for one or more parenteral administrations, and preferably by intravenous (into a vein), intravascular (in a blood vessel), intra-arterial (in an artery), intradermal (in the dermis), subcutaneous (under the skin), intramuscular (in muscle), intraperitoneal (in the peritoneum), or intratumoral (in a tumor). It can be administered as a single bolus dose, or via a continuous infusion pump.

Preferably, the microbubble, composition, kit, or any combination thereof, is formulated for administration by intravenous infusion.

Administrations can use conventional syringes and needles (e.g. Quadrafuse injection needles) or any compound or device available in the art capable of facilitating or enhancing the delivery of a microbubble to the subject (e.g. electroporation to facilitate intramuscular administration). An alternative is the use of a needle-free injection device (for example, the Biojector™ device). Transdermal patches may also be considered.

Multiple doses within the indicated ranges may be administered to the subject. In the case of repeated administration over several days or more, treatment will generally be maintained until the appearance of an observable clinical benefit. These doses may be administered intermittently, e.g. every day, every 2 or 3 days, every week, every 2 weeks, every 3 weeks or every month (e.g., so that the subject receives from about two to about twenty doses of the composition). Doses can also be adapted to each administration (for example, one or more higher initial doses followed by one or more lower doses).

In one embodiment, the microbubble, composition, kit, or any combination thereof, is administered according to a “prime boost” approach which comprises sequential administrations of one or more priming compositions and one or more boosting compositions. Typically, the priming and boosting compositions may use the same active agent (i.e. microbubble, composition, kit, or any combination thereof), or may use a different active agent (i.e. microbubble, composition, kit, or any combination thereof). In addition, the priming and reinforcing compositions can be administered to the same or a different area of the body, by the same or different routes of administration. A preferred priming and boosting approach involves a first injection (e.g. subcutaneous, intramuscular, intradermal, intratumoral or intravenous) (priming) followed by a second injection (e.g. subcutaneous, intramuscular, intradermal, intratumoral or intravenous) after an optimal period of time. The present invention encompasses one or more administrations of the priming and/or reinforcing composition(s), with preference for subcutaneous, intramuscular, intradermal, intratumoral, intranasal and intravenous routes. The time period between priming and boosting administrations varies from one week to 6 months, with a preference for one week to one month and even more for one to two weeks.

The microbubble, the composition, the kit, or any combination thereof, is (are) preferably administered in combination with the application of ultrasound, preferably the localized application of ultrasound, preferably the localized application of ultrasound on/to the area to be treated and/or marked. The ultrasound is preferably administered at a frequency ranging from 1 kHz to 10 MHz, preferably from 10 kHz to 9 MHz, preferably from 50 KHz to 8 MHz, preferably from 100 kHz to 7 MHz, preferably from 150 kHz to 6 MHz, preferably from 200 kHz to 5 MHz, preferably from 300 kHz to 4 MHz, preferably from 400 kHz to 3 MHz, preferably from 500 kHz to 2 MHz, preferably from 750 kHz to 1.5 MHz, preferably from 0.8 MHz to 1.2 MHz, even more preferably at a frequency of around 1 MHz.

The ultrasound is preferably pulsed at a frequency ranging from 1 Hz to 10 kHz, preferably from 10 Hz to 9 kHz, preferably from 50 Hz to 8 kHz, preferably from 100 Hz to 7 kHz, preferably from 150 Hz to 6 kHz, preferably from 200 Hz to 5 kHz, preferably from 300 Hz to 4 kHz, preferably from 400 Hz to 3 kHz, preferably from 500 Hz to 2 kHz, preferably from 750 Hz to 1.5 kHz, preferably from 0.8 kHz to 1.2 kHz, even more preferably at a frequency of around 1 kHz.

Ultrasound is preferably administered at an acoustic pressure ranging from 100 to 800 kPa (negative peak), preferably from 200 to 700 kPa, more preferably from 300 to 600 kPa, more preferably from 400 to 500 kPa.

Ultrasound is preferably administered for 30 to 300 seconds, preferably for 40 to 250 seconds, preferably for 50 to 200 seconds, preferably for 60 to 180 seconds, preferably for 80 to 150 seconds, preferably for 90 to 120 seconds.

The area to be treated and/or marked can be any part of the body of the subject to be treated and/or marked. Said area is preferably chosen from difficult-to-access zones, such as the central nervous system, vessels, and the tumor microenvironment. Indeed, the Inventors have notably shown that, surprisingly, lipid microbubbles according to the invention are capable of actively crossing vessels, the blood-brain barrier (BBB), or even the tumor microenvironment. The Inventors have also demonstrated that the localized application of ultrasound enables these optimized microbubbles to be targeted very precisely to the area to be treated. Thus, according to an advantageous embodiment, the microbubble, composition, kit, or any combination thereof, is (are) administered in combination with the localized application of ultrasound to the area(s) to be treated and/or mark the central nervous system, vessels, tumor microenvironment, or any combination thereof.

Advantageously, the method of administering the microbubble, composition, kit, or any combination thereof, comprises, or consists essentially of, or consists of, the following steps:
a) Administration of the microbubble, the composition, the kit, or any combination thereof, to a subject, by a suitable mode of administration (in particular as described above, preferably parenterally, more preferably intravenously);
b) Application of ultrasound to the area to be treated and/or marked (preferably according to the frequency, pulse, acoustic pressure and duration conditions described above).

The administration method may further comprise additional steps for preparing the microbubble, the composition, the kit, or any combination thereof, prior to administration step a); in particular when the microbubbles, the composition, the kit, or any combination thereof, is in lyophilized form; said additional steps being as follows:
(i) Optionally, suspension of the lyophilizate;
(ii) Optionally, microbubble activation;
(iii) Optionally, mix of the microbubbles with an excipient (in particular a diluent) and incubate for 0.5 to 15 minutes, preferably 1 to 10 minutes, preferably 1.5 to 8 minutes, preferably 2 to 6 minutes, preferably 1 to 5 minutes, preferably 1 to 3 minutes;
(iv) Optionally, the microbubbles are brought into contact with a therapeutic agent, a targeting agent, a marking agent, or any combination thereof; the therapeutic agent, the targeting agent, the marking agent, or any combination thereof; may be previously mixed with an excipient (notably a diluent).

According to one embodiment, the microbubbles are activated using a mechanical stirrer; e.g. with stirring between 2000 and 5000 revolutions per minute (rpm), preferably at 4000 rpm; for a period of between 10 and 120 seconds, preferably for 45 seconds.

According to one embodiment, when the microbubble, composition, kit, or any combination thereof, is used for marking purposes, the method further comprises a step of detecting the presence or absence of the marking agent.

### In Vitro Applications and Methods

The data reveals that the microbubble according to the invention can be effectively used as a marking, detection and imaging tool.

The present invention therefore provides efficient and reliable diagnostic methods. In particular, it enables the diagnosis, prognosis, stratification or monitoring of diseases, or the evaluation of treatment efficacy.

The present invention therefore relates to the in vitro use of at least one microbubble according to the invention (as defined above); or of a (notably pharmaceutical) composition or kit comprising, or consisting essentially of, at least one microbubble according to the invention (as defined above); or of a (notably pharmaceutical) composition as defined above; or of a kit as defined above; or of any combination thereof; for:
i. delivery of an agent (therapeutic, targeting, marking, or any combination of these) inside a cell or biological sample;
ii. marking of a cell or biological sample;
iii, the diagnosis of a disease, in a subject likely to suffer from a disease;
iv. follow-up care for a subject suffering from a disease;
v. stratification of a subject suffering from a disease;
vi. evaluating the efficacy of a treatment (particularly curative) administered to a subject suffering from a disease;
vii. detecting the presence or absence of at least one microbubble in a sample, in particular a biological sample;
viii. determine the presence or absence, or the quantity, of at least one marking agent (especially a contrast agent) in a sample, especially a biological sample;
ix. screening for compounds/molecules with an effect in the prevention, treatment, marking, or any combination thereof, of a disease; or
x. any combination of i. to ix.

The present invention relates in particular to the in vitro use of at least one microbubble according to the invention (as defined above); or of a composition (in particular pharmaceutical) or of a kit comprising, or consisting essentially of, at least one microbubble according to the invention (as defined above); or of a composition (in particular pharmaceutical) as defined above; or of a kit as defined above; or of any combination thereof; to deliver an agent inside at least one cell, or a biological sample, preferably by sonoporation;
the agent being preferably selected from a therapeutic agent, a targeting agent, a marking agent, and any combination thereof;
the cell being preferably selected from an animal cell, a plant cell, a microorganism cell, and any combination thereof.

The microbubble, composition, kit, or any combination thereof, is (are) preferably brought into contact with the cell or biological sample (especially administered to the sample) in combination with the application of ultrasound, preferably the localized application of ultrasound, preferably the localized application of ultrasound on/to the area to be treated and/or marked. The ultrasound is preferably applied at a frequency ranging from 1 kHz to 10 MHz, preferably from 10 kHz to 9 MHz, preferably from 50 KHz to 8 MHz, preferably from 100 kHz to 7 MHz, preferably from 150 kHz to 6 MHz, preferably from 200 kHz to 5 MHz, preferably from 300 kHz to 4 MHz, preferably from 400 kHz to 3 MHz, preferably from 500 kHz to 2 MHz, preferably from 750 kHz to 1.5 MHz, preferably from 0.8 MHz to 1.2 MHz, even more preferably at a frequency of around 1 MHz.

The ultrasound is preferably pulsed at a frequency ranging from 1 Hz to 10 kHz, preferably from 10 Hz to 9 kHz, preferably from 50 Hz to 8 kHz, preferably from 100 Hz to 7 kHz, preferably from 150 Hz to 6 kHz, preferably from 200 Hz to 5 kHz, preferably from 300 Hz to 4 kHz, preferably from 400 Hz to 3 kHz, preferably from 500 Hz to 2 kHz, preferably from 750 Hz to 1.5 kHz, preferably from 0.8 kHz to 1.2 kHz, even more preferably at a frequency of around 1 kHz.

Ultrasound is preferably administered at an acoustic pressure ranging from 100 to 800 kPa (negative peak), preferably from 200 to 700 kPa, more preferably from 300 to 600 kPa, more preferably from 400 to 500 kPa.

Ultrasound is preferably administered for 30 to 300 seconds, preferably for 40 to 250 seconds, preferably for 50 to 200 seconds, preferably for 60 to 180 seconds, preferably for 80 to 150 seconds, preferably for 90 to 120 seconds, preferably for 60 seconds.

Advantageously, the method of administering the microbubble, composition, kit, or any combination thereof, comprises, or consists essentially of, or consists of, the following steps:
a) contact of the microbubble, the composition, the kit, or any combination thereof, with a cell or a biological sample; or administration of the microbubble, the composition, the kit, or any combination thereof, a biological sample, by an appropriate mode of administration (in particular as described above, in the “Therapeutic Uses and Methods” section);
b) Application of ultrasound to the area to be treated and/or marked (preferably according to the frequency, pulse, acoustic pressure and duration conditions described above).

The administration method may further comprise additional steps for preparing the microbubble, the composition, the kit, or any combination thereof, prior to administration step a); in particular when the microbubbles, the composition, the kit, or any combination thereof, is in lyophilized form; said additional steps being as follows:
(i) Optionally, suspension of the lyophilizate;
(ii) Optionally, microbubble activation;
(iii) Optionally, mix of the microbubbles with an excipient (in particular a diluent) and incubate for 0.5 to 15 minutes, preferably 1 to 10 minutes, preferably 1.5 to 8 minutes, preferably 2 to 6 minutes, preferably 1 to 5 minutes, preferably 1 to 3 minutes;
(iv) Optionally, the microbubbles are brought into contact with a therapeutic agent, a targeting agent, a marking agent, or any combination thereof; the therapeutic agent, the targeting agent, the marking agent, or any combination thereof; may be previously mixed with an excipient (notably a diluent).

According to one embodiment, when the microbubble, composition, kit, or any combination thereof, is used for marking purposes, the method further comprises a step of detecting the presence or absence of the marking agent in the cell and/or biological sample.

The microbubble and its constituents, such as lipophosphoramidate, histidylated polyethylenimine, therapeutic agent, targeting agent, marking agent, biocompatible gas, are advantageously as described in the preceding sections (in the “Definition” and/or “Lipid microbubble” and/or “Methods of producing a lipid microbubble” sections).

Advantageously, the composition and/or kit are as described above in the “Compositions and kits” section.

The following examples are intended to illustrate the present invention, and should not be regarded as limiting.

### DESCRIPTION OF FIGURES

FIG. 1: Summary diagram of the formulations developed. Functionalization of the microbubble can depend on various constituents, such as cationic lipids (capable of electrostatic interactions), and biotinylated lipids (capable of binding antibodies via streptavidin-biotin bonds).

FIG. 2: Chemical structure of cationic (LIPIDE 1) and fusogenic (LIPIDE 2) lipids used in lipophosphoramidate-based formulations.

FIG. 3: Size distribution of the different microbubble formulations developed. Analysis was carried out using microscopy image analysis (ImageJ® software).

FIG. 4: Zeta potential measurements (mV) of different formulations. Values represent the mean±SD of 3 measurements.

FIG. 5: Size distribution of developed microbubble formulations, including KLN25.

FIG. 6: Complexation gel (0.6% agarose) of different volumes of MBc (cationic MB) with a constant amount of plasmid DNA (1 μg).

FIG. 7: Confocal imaging of MBc-tPTX gas microbubbles complexing FITC-coupled CpG oligonucleotides.

FIG. 8: Schematic diagram of the set-up used for flow analysis of functionalized MB targeting.

FIGS. 9A-C: FIG. 9A) Graph showing MB44 binding to hCMEC/D3 cells stimulated or not for VEGF receptor production. Tracks: dash-dotted line) MBa on stimulated cells; dotted line) MBa carrying anti-VEGFR2 receptor on unstimulated cells; solid line) MBa carrying anti-VEGFR2 receptor on stimulated cells. FIG. 9B) Graph showing MBc-tPTX binding to cells stimulated to produce the receptor for 24 h. Tracks: dotted line) MBc-tPTX without antibody; solid line) MBc-tPTX carrying antibody. FIG. 9C) Graph showing the binding of MBc-tPTX complexing CpG ODNs to cells stimulated to produce the receptor for 48 h. Tracks: dotted line) MBc-tPTX without antibody; solid line) MBc-tPTX with antibody. Vertical bar: end of injection, beginning of rinses.

FIG. 10: Scheme of the in vivo sonoporation experimental set-up, comprising a focused ultrasound probe and a motorized positioning system. The mouse is placed in the cradle after injection of Evan's Blue I.V. the left hemisphere is then targeted, USF treatment is performed 10 s after injection of MBs for 60 s. Adapted from Celia Si Ahmed, M2 2018, Orléans.

FIGS. 11A-C: FIG. 11A) Scheme showing brain partitioning for luciferase activity assay (blue: USF-treated area). FIG. 11B) Photograph of the brain of a mouse treated with MBc+USF at 109 kPa, 24H post-transfection, the blue spot corresponds to the extravasation of Evan's blue. FIG. 11C) Photograph of a brain included for Cryostat® sectioning.

FIG. 12: Graph showing luciferase activity per mg protein in different brain zones 24 h after sonoporation with MBc-complexed pLuc plasmid. The area targeted by the ultrasound device is zone 2. Data represent mean±SEM. **: p<0.01.

FIGS. 13A-C: Microscopic observation of anionic (MB1) and cationic (MBPEI) microbubble suspensions. Objectives 25. FIG. 13A) MBI suspension after activation in HEPES (10 mM, pH 7.4, filtered at 0.2 nm). FIG. 13B) MBPEI suspension after activation in HEPES (10 mM, pH 7.4, filtered at 0.2 nm). FIG. 13C) MBPEI flocculation during preparation of MB: pDNA complexes for in vivo manipulations. MBI-Size & concentration (n=6); MBPEI-Size & concentration (n=8).

FIGS. 14A-C: Fluorescence microscopy 24 h after transfection of the eGFP-encoding plasmid using MBPEIhis on a HeLa cell line. FIG. 14A) Variation in MB: DNA ratio over an amplitude of 431 kPa.

FIG. 14B) Variation in MB: DNA ratio in the absence of US. FIG. 14C) Acoustic pressure/amplitude variation over a stable MB: DNA ratio (1:2).

### EXAMPLES

### Example: Design, Development and Properties of Microbubbles for Targeted Delivery of Active Ingredients

### Introduction

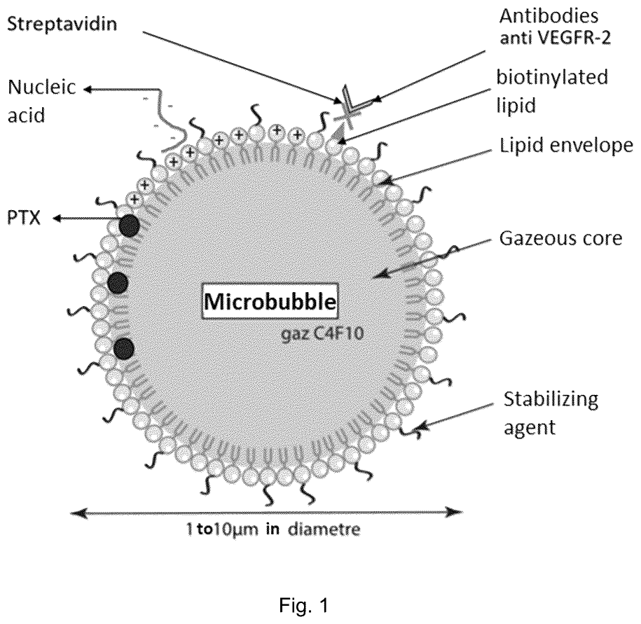

In the context of the present invention, a new formulation of gas microbubbles has been developed. These microbubbles are particularly advantageous, since they can both encapsulate different types of active ingredients, such as nucleic acids, and deliver them in a localized manner after activation by focused ultrasound (FIG. 1). A new device has also been developed, enabling ultrasound to be delivered to the mouse brain in a targeted manner. This system is coupled to these original gas microbubbles for encapsulation and delivery of active ingredients. The formulation of gas microbubbles is activated and then, using a stirrer, the active ingredient can be present in the formulation or can be added after the formulation has been activated. The device is motorized and positioned at the coordinates corresponding to the user's desired delivery site. The original microbubbles developed here are capable of crossing the BBB. In this way, the positioning system developed can be implemented with a brain atlas to localize the brain structures to be treated. Gas microbubbles are injected systemically, followed by ultrasound. The device can be fitted with a passive cavitation detection system to monitor ultrasound activation of the microbubbles in real time.

Microbubble gas formulations can be used to encapsulate or co-encapsulate various active ingredients:
(i) liposoluble active ingredients, such as chemotherapeutic agents (e.g. paclitaxel), notably on the surface and/or in the envelope of the microbubble;
(ii) anionic actives such as nucleic acids (plasmid DNA, small RNAs, messenger RNAs, etc.; e.g. for gene therapy), for example using cationic lipids;
(iii) antibodies, for example using the streptavidin-biotin couple, or chemistry-click, or lipids coupled to methyl tetrazine groups;
(iv) proteins or peptides (such as cell-penetrating peptides (CPPs)), e.g. using streptavidin-biotin, chemistry-click or lipids coupled with methyl tetrazine groups.

This high degree of flexibility is due in particular to the original formulation of the microbubbles, which uses a mixture of cationic molecules such as lipophosphoramidates and/or histidylated polyethyleneimine.

Indeed, two types of cationic microbubbles have been developed: one based on lipophosphoramidates and the other on histidylated polyethyleneimine coupled to a fatty acid.

These microbubbles have already demonstrated their efficacy in vitro and in vivo as ultrasonic theranostic agents (therapy and imaging). The blood-brain barrier was transiently permeabilized without danger to the animal, this was validated by MRI imaging and histology. Expression of a luciferase transgene was detected after the sonoporation protocol.

### Example 1: Microbubbles Comprising Lipophosphoramidates

### 1.1 Materials and Methods

### 1.1.1. Production of Microbubbles (MB) According to the Invention

The first step in MB production is to mix different lipids according to the type of microbubble required.

1.1.1.1. MB with KLN27

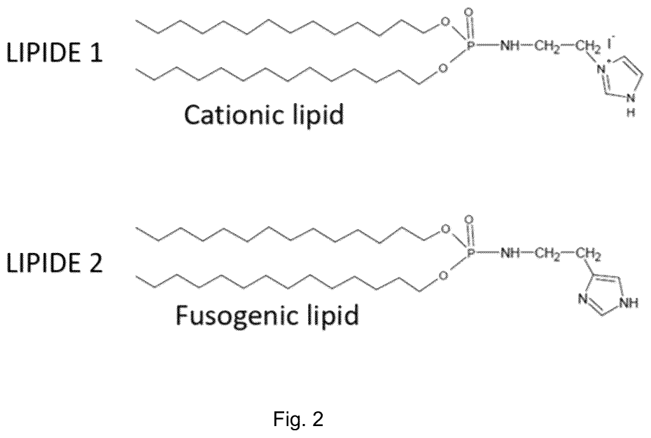

The KLN27 and MM30 lipids (FIG. 2) come from a collaboration with the University of Brest (Berchel). These are phosphoramidate lipids, LIPIDE1 (KLN27) is cationic in nature, while LIPIDE2 (MM30) is used as a fusogenic lipid (a lipid that can fuse to membranes, notably used to promote endosomal escape). All other lipids used (DMPC, DSPC, DMPE-PEG2000, DSPE-PEG2000, DSPE-PEG2000biot) were sourced from Avanti Polar Lipids (Alabaster, AL, USA). The various lipids are mixed in a flask in the presence of absolute ethanol (99.96% pure). During this step, PTX solubilized in absolute ethanol (10 mM, Merck, Germany) is added according to the formulation (Table 2).

TABLE 2
List of formulations used. The proportions of the various lipids making up the envelope
are indicated in molar percentage, relative to the total molar quantity of lipids.

| | | | Nature | | | PEGylated lipid | | | Cationic | Fusiogenic | |
|---|---|---|---|---|---|---|---|---|---|---|---|
| | | | of the | | | | | DSPE- | lipid | lipid | Other |
| | | | lipid | Neutral Lipid | | DSPE- | DMPE- | PEG2000 | LIPIDE | LIPIDE | PTX |
| | | | Name | DSPC | DMPC | PEG2000 | PEG2000 | biot | 1 | 2 | (μM) |
| Cationic | Not | Control | MBc | | 22.5% | | 10% | | 45% | 22.5% | |
| | targeted | PTX | MBc- | | 22.5% | | 10% | | 45% | 22.5% | 175.66 |
| | | | PTX | | | | | | | | |
| | Targeted | Control | MBc-t | | 22.5% | | 9% | 1% | 45% | 22.5% | |
| | (t) | PTX | MBc- | | 22.5% | | 9% | 1% | 45% | 22.5% | 175.66 |
| | | | tPTX | | | | | | | | |

| | | | | |
|---|---|---|---|---|
| Targeted Anionics | MBa | 90% | 9% | 1% |

The mixture is then evaporated using a Rotavapor (Büchi, Schwabach, Germany) for 30 min, 20 rpm at 60° C. After this stage, a lipid film forms in the flask. The lipid film formed is taken up in 2 mL HEPES (10 mM, pH 7.4) and sonicated for 5 min to obtain a homogeneous lipid solution (FIGS. 11A-C). The solution is then distributed in equal volumes into 4 crimp vials (VWR International, Radnor, PA, USA), which are stored for 1 h at −80° C. Finally, the vials are placed in a freeze-dryer (Bioblock Scientific, Illkirch, France) overnight. Once the lyophilizate has been recovered, the vials are crimped by hand and stored at 4° C. before activation. Activation involves replacing the air in the flask with perfluorobutane (C4F10, F2 Chemical, UK) by overpressure, then rehydrating the lyophilizate with 500 μl of 10 mM HEPES solution. The flasks are agitated for 45 s using a VIALMIX (Bristol Myers Squibb, USA). This step allows microbubbles to form in the vial, but it is necessary to wait 5 min post-agitation before sampling. Once activated, the vial can be stored for a few days at 4° C.

1.1.1.2. MB with KLN25

MB comprising the phosphoramidate lipid KLN25 are also prepared as described above (paragraph 1.1.1.1).

The proportions of the various lipids making up the envelope in molar percentage, relative to the total molar quantity of lipids, are as follows:
47.5% KLN25;
47.5% MM27; and
5% DSPE-PEG (5000).

### 1.1.1.3. MB Comprising Dipalmitoyl Phosphoramidates

MB comprising dipalmitoyl phosphoramidate lipids are also prepared as described above (paragraph 1.1.1.1).

The proportions of the various lipids making up the envelope in molar percentage, relative to the total molar quantity of lipids, are as follows:
47.5% dipalmitoyl phosphoramidate;
47.5% MM27; and
5% DSPE-PEG (5000).

### 1.1.1.4. MB Comprising Disteraoyl Phosphoramidates

MB comprising disteraoyl phosphoramidate lipids are also prepared as described above (paragraph 1.1.1.1).

The proportions of the various lipids making up the envelope in molar percentage, relative to the total molar quantity of lipids, are as follows:
47.5% disteraoyl phosphoramidate;
47.5% MM27; and
5% DSPE-PEG (5000).

### 1.1.2. Anionic MB (Comparative Example)

Anionic microbubbles (MBa) are produced using the method described in paragraph 2.1.1 below.

### 1.1.3. Characterization of Microbubbles

### 1.1.3.1. Concentration and Size

Microbubbles are observed using an inverted microscope (Nikon Diaphot 300 invert) connected to a computer. Photographs are taken using a FASTCAM SA7 camera (Photron, USA) and ICcapture® software. Photos are taken with different lenses (×10, ×20, ×40) and then processed by ImageJ®. Processing consists of 8-bit image conversion followed by thresholding to detect MB contours. Particle analysis is then used to count the number of particles and obtain their size. Microbubbles are diluted to the 10th or 100th in HEPES (10 mM, pH 7.4) and then deposited on a Malassez slide.

### 1.1.3.2. Measurement of Zeta Potential (ζ)

The ζ potential corresponds to the overall charge of a particle at its shear plane (particle surface). It is measured using the Nano partica SZ-100 (Horiba, Japan). To perform the measurements, 30 μL of MB are diluted in 970 μL of 10 mM HEPES pH 7.4. Analysis is performed at 25° C.

### 1.2 Results

### 1.2.1. Characterization of Microbubbles According to the Invention and Comparison with Anionic Microbubbles

The size and concentration of microbubbles prepared according to the present example (MB according to the present invention, prepared as described in paragraph 1.1.1 above) are assessed by optical imaging.

1.2.1.1. MB with KLN27

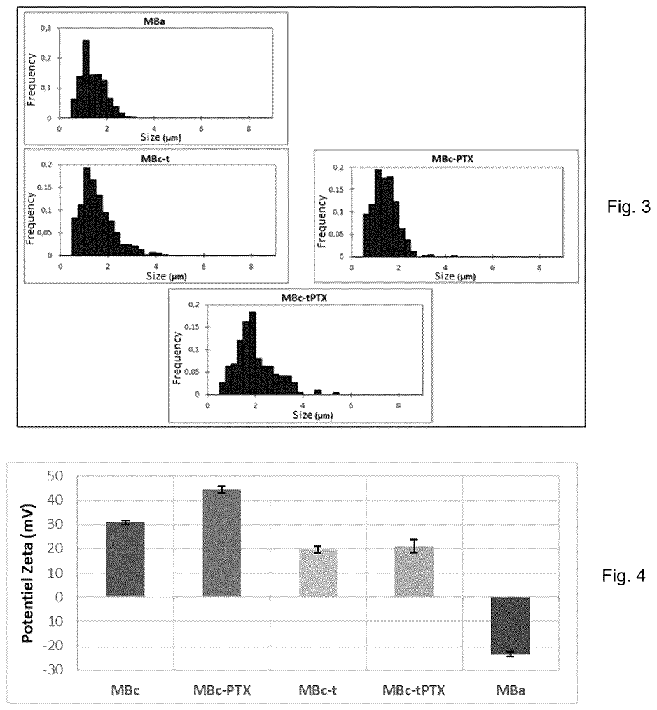

FIG. 3 shows the results obtained for the different formulations in Table 2. The size information collected shows an average diameter of 1.41 μm for MBc and 1.41 μm for MBc-PTX, while for MBc-t it is 1.55 μm. MBc-tPTX have an average diameter of 1.98 μm. As for anionic microbubbles, anionic MB (MBa) have an average size of 1.41 μm. The concentrations of the different formulations are shown in Table 3. The distribution in size and concentration remains homogeneous between the two main categories of microbubbles (cationic and anionic). Generally speaking, anionic MB (similar to commercial MB) are similar in size to cationic MB, but have a higher concentration. MBc functionalized with PTX appear slightly smaller and in higher concentration than the base formulation (MBc). MBc-tPTX with both biotinylated lipids and PTX had the largest size and lowest concentration. However, the size distribution of MBs remains below 10 μm, enabling them to be injected in vivo.

TABLE 3
Summary table of average concentrations
and sizes of different MB formulations.

| | | Average | |
|---|---|---|---|
| | | diameter | |
| MB | Formulation | (μm) | Concentration (MB/mL) |
| Cationic | MBc | 1.41 | 8.3 × 10⁸ |
| | MBc-PTX | 1.41 | 9.3 × 10⁸ |
| | MBc-t | 1.55 | 1.0 × 10⁹ |
| | MBc-tPTX | 1.98 | 4.25 × 10⁸ |
| Anionic | MBa | 1.41 | 2.38 × 10⁹ |

FIG. 4 shows the ζ potential measurements of the developed formulations. These results show no significant change in overall charge when MBc are functionalized with PTX, biotin, or both at the same time. These remain positive (average MBc: +28.8 mV). The MBa formulation has no KLN27 (cationic lipid) and therefore serves as a control, with a charge of −23.4 mV.

1.2.1.2. MB with KLN25

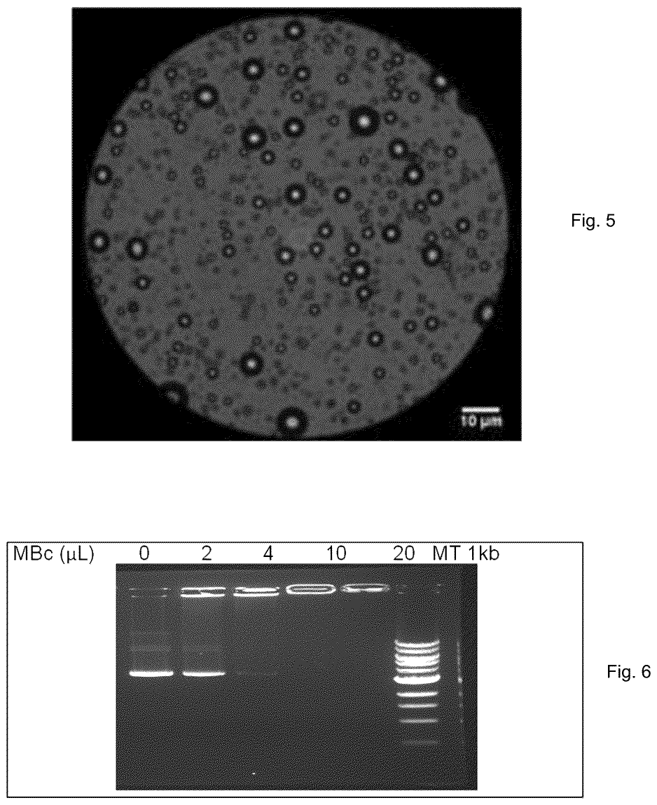

FIG. 5 shows the results obtained for KLN25-containing formulations, confirming the possibility of forming KLN25-containing MB. The size and concentration distribution of MB containing KLN25 is comparable to that of MBs containing KLN27.

### 1.2.1.3. MB Comprising Dipalmitoyl Phosphoramidates or Disteraoyl Phosphoramidates

The results obtained for formulations comprising dipalmitoyl phosphoramidates or disteraoyl phosphoramidates are comparable to those obtained with formulations comprising KLN27 or KLN25. The size and concentration distribution of MB comprising dipalmitoyl phosphoramidates or disteraoyl phosphoramidates is comparable to that of MB comprising KLN27 or KLN25. These data confirm the possibility of forming MB from dipalmitoyl phosphoramidates or disteraoyl phosphoramidates.

### 1.2.2. Complexation of Nucleic Acids

The ability of microbubbles to vectorize nucleic acids is assessed by gel retardation. FIG. 6 shows the complexation capacity of MBc microbubbles (possessing the cationic lipid KLN27) in the presence of 1 μg plasmid DNA (plasmid pLuc). When no microbubbles are present, a band migrating at around 3 kilobases appears after UV revelation. This band corresponds to uncomplexed plasmid DNA. As MB concentration increases, the intensity of the band at 3 kb decreases, corresponding to complexation of pDNA with microbubbles. When pDNA is complexed, it no longer migrates and remains in the deposition wells. When 10 μL of MB is used, all the pDNA is complexed, and the 3 kb band is no longer visible, but rather marked in the wells.

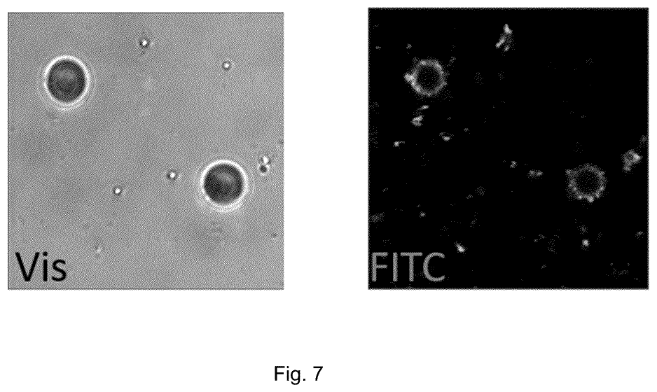

This complexing ability was also confirmed by confocal fluorescence microscopy (FIG. 7). MBc-tPTX are complexed for 2 min with FITC-coupled CpG oligonucleotides at a ratio of 1 μg nucleic acid to 10 μL microbubbles. Fluorescence around the microbubbles confirms nucleic acid complexation. Fluorescent debris is visible around the microbubbles, probably indicating the formation of lipoplexes from fragments of destroyed microbubbles or free lipids in solution.

### 1.2.3. Assessment of Functionalized MB Targeting

To assess the ability of MB to target the VEGF receptor, hCMEC/D3 cells stimulated to produce VEGFR are cultured in Ibidi®, followed by flow analysis of MB using light microscopy. 6-channel flow culture plates (Ibidi® u-Slide VI0.4, Clinisciences) are seeded with 18,000 cells/channel.

Once the cells have attached to the bottom of the channels, the wells are filled with culture medium supplemented (or not) with TGFβ at 5 ng/ml, and the cells are incubated for 24 h or 48 h at 37° C. in a 5% CO₂ atmosphere. The optimal TGFβ concentration for increasing the number of VEGFR2 receptors on the endothelial cell surface is determined by flow cytometry.

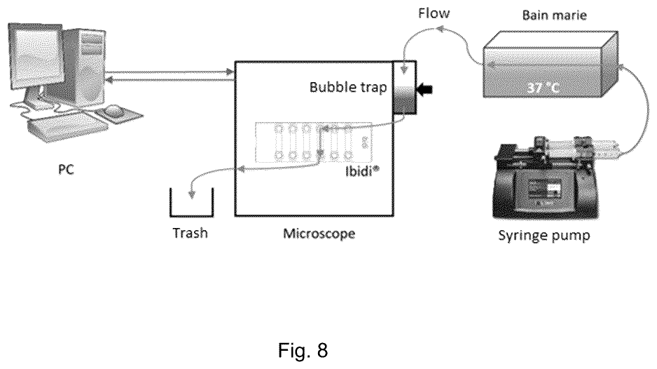

The Ibidi® plate is then positioned on an inverted microscope equipped with a camera and connected to the computer. The set-up used throughout the experiment is shown in FIG. 8.

To analyze microbubble attachment, a field of view is selected. Different types of microbubbles are used (described in Table 2 above). Following flow cytometry analysis, some microbubbles are functionalized with the antibody directed against the VEGF receptor (VEGFR2). To this end, 1.23 μL of streptavidin (15 mM) are incubated in the presence of 0.3465 μL of anti-VEGFR2-Biot antibody (Anti-mouse CD309, 0.5 mg/mL, eBioscience) to obtain a ratio of 2.5 moles of antibody per mole of streptavidin. Next, 20 μL of the MB solution is incubated for 10 min to functionalize the MB, preventing the formation of microbubble aggregates by streptavidin-biotin bonds. At the end of the 10 min, a solution of 10 mM HEPES pH 7.4 is added to reach a final volume of 1 mL.

The rest of the experiment consists of a succession of washes of the cultured cells. First, a 10 min wash with PBS takes place, at a flow rate of 0.137 mL/min (0.25 dyn/cm²). Next, the MB contained in 1 mL is injected for 15 min at the same flow rate, followed by a first rinse for 10 min with PBS, then a second rinse for 10 min at 0.275 mL/min (0.5 dyn/cm²), finishing with a third rinse for 10 min at 0.550 mL/min (1 dyn/cm²), followed by a final rinse at 1.1 mL/min (2 dyn/cm²). Photographs of the channel are taken every minute after injection. The series of photos is then processed in ImageJ® to obtain a count of the number of MB per minute.

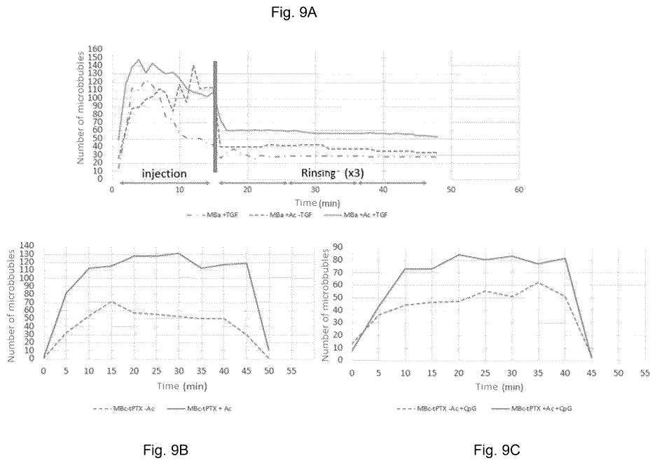

The ability of microbubbles to target the VEGF receptor (VEGFR-2) was assessed in vitro in real time using a flow-through culture chamber. The cells used for this analysis were hCMEC/D3 endothelial cells, stimulated or not by TGFβ. Different formulations were tested on these cells to assess the effect of different constituents on microbubble attachment capacity (FIGS. 9A-C).

FIG. 9A shows the results obtained for the anionic formulation MBa. The volume of MB injected is the same for each condition tested. This formulation is tested without antibody on 24 h stimulated cells (dash-dotted line), with antibody on unstimulated cells (dotted line) and with antibody on 24 h stimulated cells (solid line). At t=15 min, the 1st rinse starts. The results show a very low number of bound MB on the dashed curve, corresponding to non-specific interactions of these MB on the cells, with 42 MBa bound at t=15 min. This number drops to 27 at the end of the washes. Concerning the dotted curve, corresponding to specific binding of MB to cells, the number of MB bound is 112 at t=15 min. This number decreases to 33 at the end of the washes. The solid curve, corresponding to cells stimulated to produce VEGFR2 in the presence of targeted MB, shows a number of bound MB at t=15 min of 102. This number decreases to 52 at the end of the washes. This curve therefore records the highest number of attachments over time.

FIGS. 9B and 9C show the results obtained for the cationic formulation MBc-tPTX (incorporating PTX and biotinylated lipids). This formulation is tested on cells stimulated at 24 h and 48 h. Injection and washing speeds are halved compared with FIG. 9A, so as not to stress the cells too quickly. When cationic microbubbles are functionalized with the anti-VEGFR2 antibody, they bind in greater numbers than microbubbles not functionalized for the antibody (FIGS. 9B, 9C). Without nucleic acid complexation, the number of antibody-fixed MB is around 110% higher than the number of antibody-free MB. Complexation of CpG oligonucleotides (FIG. 9C) by microbubbles reduces receptor binding capacity. When the microbubble contains both antibody and nucleic acid, the number of bound MB is around 60% higher than in the control without antibody.

### 1.2.4. In Vivo Experimentation

### 1.2.4.1. In Vivo Sonoporation

In order to perform MB and USF transfection, the hair on the mouse's skull must be removed to allow perfect transmission of US between the skin and the ultrasound probe. Once the mouse has been prepared, it is anesthetized with an oxygen/air mixture of 1.5% isoflurane (Vetflurane, France) throughout the experiment. A catheter equipped with a 26 G needle is placed in the tail vein for intravenous injections. An injection of Evan's Blue (5%, 1 mL/kg saline solution) is performed. Evan's blue is a dye that binds to circulating albumin and does not naturally cross the BBB. However, its extravasation is visible after opening via MB+USF, enabling us to obtain information on the location of the area targeted by our protocol once the brain has been extracted from the skull.

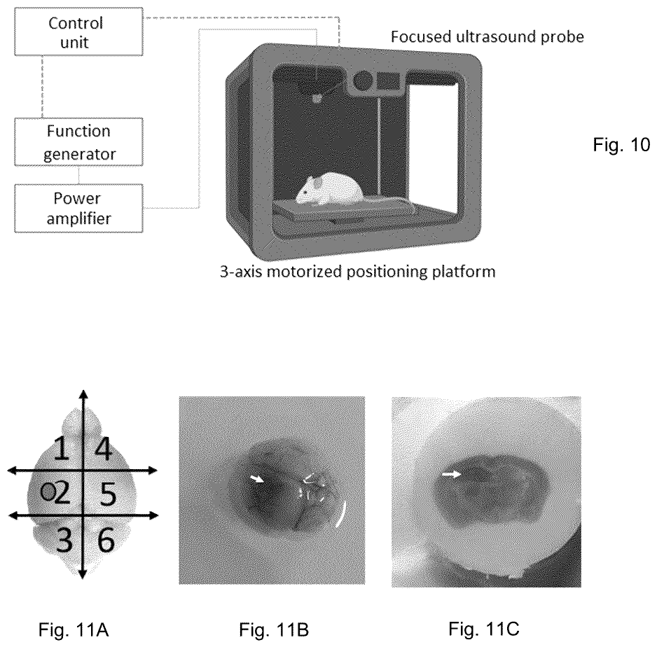

The mouse is then placed in the cradle of the in vivo sonoporation platform (FIG. 10). USF is applied using a 54 mm diameter single-element focused ultrasound transducer (Precision Acoustics, UK). The transducer is placed in a sealed cylinder filled with degassed water and sealed by a membrane. Bubbles should be avoided to prevent poor US propagation. The transducer is positioned on a motorized control platform connected to a computer, enabling it to be moved using Repetier software. The transfection zone is targeted visually using a pointer located on the transducer. The chosen zone corresponds to half the eye-ear distance in the left hemisphere (zone 2). Ultrasonic conduction gel is applied to the mouse's skull to enable US transmission. After aiming, the transducer is positioned over the aimed zone and then descends to contact the gel.

A solution of 30 μg pDNA diluted in 60 μL 10 mM HEPES pH 7.4 and 20 μL 20% sucrose is then prepared. This solution is incubated for 2 min in the presence of 120 μL MBc and then injected intravenously into the mouse. Ten seconds after the end of the injection, the US was sent for 60 s (1 MHz, 5% duty cycle, pulse duration 1 sec). Two acoustic pressures were tested: 109 kPa and 145 kPa. After US application, the mice were awakened.

### 1.2.4.2. Measurement of Luciferase Activity (RLU Assay)

24 hours after sonoporation, mice transfected with the plasmid encoding luciferase are euthanized and their brains harvested. The brain is sectioned into 6 parts: zones 1, 2 and 3 correspond to the left side of the brain, and zones 4, 5 and 6 to the right side. The transfection zone targeted by our protocol is located in zone 2, visualized by Evan's blue extravasation (FIGS. 11A-C).

Each section is then placed in Eppendorf® tubes and immersed in liquid nitrogen. The sections are then crushed manually in a mortar in the presence of liquid nitrogen, the crushed material is then solubilized in 500 μL of CCLR lysis buffer for 1 h30. Centrifugation is carried out (5 min, 12,000 RPM, 4° C.), and 150 μL of supernatant is collected for reading on a Lumat LB9507 luminometer (Berthold, Germany). The instrument is then loaded with a solution of LAR (Luciferase Assay Reagent) to measure luciferase activity. The rest of the lysate is then used to determine the protein concentration contained in each zone. This quantification is performed using a BCA assay. The values obtained are then normalized with luciferase activity values in RLU/mg protein.

The efficiency of nucleic acid delivery was tested by measuring the activity of a reporter gene encoding luciferase. This gene was transfected into the brain using cationic microbubbles (MBc) and focused ultrasound. Two different acoustic pressures were tested, 109 kPa (n=8) and 145 kPa (n=5).

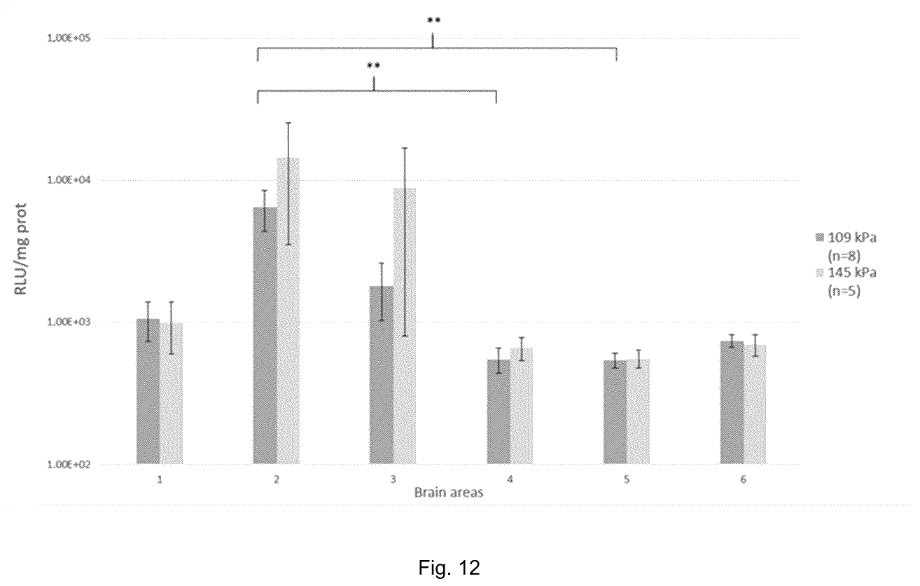

The results presented in FIG. 12 show strong luciferase activity (between 5.5×10³ and 1.05×10⁴ RLU/mg protein) in zones 2 and 3 of the mouse brain, for the two ultrasound powers used. Luciferase expression was significantly different between zones 2 and 4, and for 2 and 5 at 109 kPa. Results obtained at 145 kPa were not significantly different from those obtained at 109 kPa. Luciferase activity is low in brain zones 1, 4, 5 and 6 (below 10³ RLU/mg protein). It should be noted that these values are close to those of the background of non-transfected tissue. As targeting is not stereotactic, and zoning is imprecise, zones 1 and 3 may show luciferase activity. High luciferase expression is therefore mainly measured in the left hemisphere (where targeting takes place) and not in the right, confirming the possibility of using focused ultrasound coupled with cationic microbubbles for nucleic acid delivery.

### 1.2.4.3 Immunohistochemistry

Brain sections from mice transfected with different plasmids were cut to study the area of transfection and the cell types involved. Transfection zones were determined by observing the extravasation of Evan's Blue on the section.

### Example 2: Microbubbles Comprising Histidylated Polyethylenimines (MBPEI)

### 2.1. Materials and Methods

### 2.1.1. Production of Anionic Microbubbles (Comparative Example)

The production of anionic microbubbles (MBa) involves several steps. The first involves mixing phospholipids in a 50 mL flask. For this formulation of MB, distearoylphosphatidylcholine (DSPC) is mixed with 1,2-distearoyl-sn-glycero-3-phosphoethanolamine-N-[methoxy (polyethylene glycol)-2000] (DSPE) at a ratio of 90%-10% to 87%-13% (molar ratio) depending on the batch of MB, in a final volume of 2 mL of 99.9% ethanol. The solution is then heated to 60° C. under vacuum in a Rotavapor (Buchi, Schwabach, Germany), until a lipid film is formed in the flask. This film is then resuspended in 2 mL HEPES (10 mM, pH 7.4, filtered with a 0.2 μm filter), and sonicated for 5 min in a sonication bath (35 kHz). This solution is then dispensed into four glass vials and stored for 1 h at −80° C. Finally, the vials are lyophilized (Bioblock Scientific, Illkirch, France) for at least 12 h. The vials are then crimped to make them hermetically sealed, and stored at 4° C. until activation.

Vials are activated by replacing the air inside with a high-molecular-weight gas, perfluorobutane (C4Fio, F2 Chemical, UK), then adding 500 μL HEPES (10 mM, pH 7.4, filtered through a 0.2 μm filter) to the vial using a syringe. The vials are then shaken for 45 seconds using a VIALMIX (Bristol Myers Squibb, USA). MB produced in this way are called MB1.

### 2.1.2. Production of MBPEI Microbubbles According to the Invention

Cationic MB comprising histidylated polyethyleneimines are produced by a process comprising steps similar to those described in paragraph 2.1.1 above (MB1 production). Here, however, PEI (Polyethyleneimine+Stearic Acid) lipids coupled to histidine are used to obtain a positively charged envelope. The method for activating these microbubbles is similar to that for MB1s. The MBs thus produced are called MBPEIhis.

### 2.1.3. Characterization of Microbubbles

### 2.1.3.1. Concentration and Size

Microbubbles are observed using an inverted microscope (Nikon Diaphot 300 invert) connected to a computer. Photographs are taken using a FASTCAM SA7 camera (Photron, USA) and ICcapture® software. Photos are taken with different lenses (×10, ×20, ×40) and then processed by ImageJ®. Processing consists of 8-bit image conversion followed by thresholding to detect MB contours. Particle analysis is then used to count the number of particles and obtain their size. Microbubbles are diluted to the 10th or 100th in HEPES (10 mM, pH 7.4) and then deposited on a Malassez slide.

### 2.1.3.2. Evaluation of In Vitro Nucleic Acid Delivery

In vitro sonoporation experiments were carried out on HeLa and HepG2 cell lines, at 20,000 cells/well (HeLa) and 30,000 cells/well (HepG2), with plasmid DNAs encoding the GFP protein. To assess the transfection capacity of MBs in the presence or absence of US, each sonoporation condition was performed in duplicate on 48-well culture plates. Sonoporation transfections were performed in 190 μL of Opti-MEM™ medium, with 10 μL of MB/pDNA complexing solution. Each transfection condition in the presence of US was treated for 1 min. After transfection, culture plates were incubated at 37° C. under 5% CO2 for 30 min in Opti-MEM™ medium, then for 48 h in conventional culture medium at 37° C. with 5% CO2. Transfection results were analyzed by fluorescence microscopy 24 h after sonoporation.

### 2.2. Results

### 2.2.1. Characterization of MB According to the Invention and Comparison with MBa

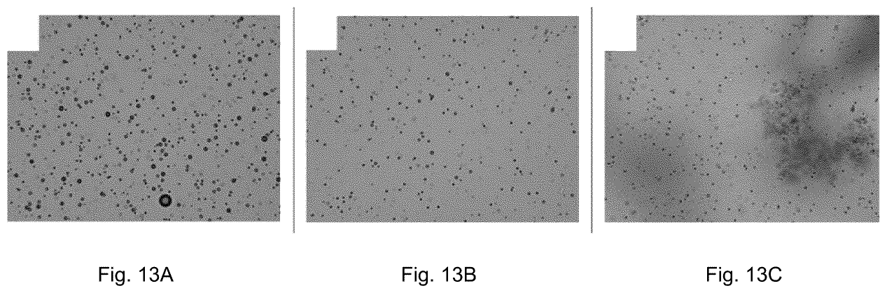

The size and concentration of MB prepared according to the present example (MBPEIhis according to the present invention, prepared as described in paragraph 2.1.2 above) are evaluated by optical imaging. FIGS. 13A-C shows the results obtained for the different formulations. The size information collected shows an average diameter of 1.55 μm for MBa and 1.31 μm for MBPEIhis. Thus, the size distribution of MBPEIhis is less than 10 μm, enabling them to be injected in vivo.

The average concentration is 2.18×10¹⁰ MB/mL for MBa and 8.51×10⁹ MB/mL 1.31 μm for MBPEIhis.

### 2.2.2. Evaluation of In Vitro Nucleic Acid Delivery

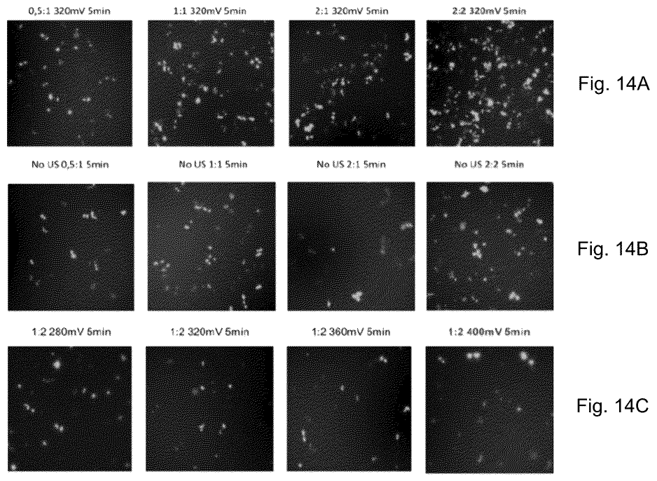

In vitro nucleic acid delivery efficiency was measured using the eGFP reporter gene. This gene was transfected onto the HeLa cell line, using MBPEIs or MB1 in the presence or absence of US. Different MB:pDNA ratios and acoustic pressures were tested. The results of this transfection were then analyzed by fluorescence microscopy, showing sonoporation transfection efficiency of MBPEIs microbubbles (FIGS. 14A-C).

### Discussion

Taken together, these data show that the innovative microbubbles developed by the present Inventors are capable of transporting different types of drugs, including nucleic acids, stably in the bloodstream; crossing the blood-brain barrier (BBB); and delivering drugs in a targeted manner, particularly towards antigens of interest.

In particular, the Inventors have shown that, surprisingly, the lipid microbubbles developed in this way possess significantly improved stability, unlike the microbubbles described in the prior art. Remarkably, the data also reveal that these optimized microbubbles are capable of delivering different types of agents of interest, including nucleic acids, more efficiently than microbubbles described in the prior art. Particularly interestingly, these microbubbles are able to cross vessels as well as the BBB. The Inventors have also demonstrated that the localized application of ultrasound enables these optimized microbubbles to be targeted very precisely to the area to be treated. These data thus reveal the therapeutic potential of these lipid microbubbles to treat a wide range of pathologies in a targeted manner, including central nervous system pathologies, vessel pathologies, cancers and tumors.

The data also show that these optimized microbubbles are effective detection and imaging tools. The present invention therefore provides both powerful, broad-spectrum treatment methods for pathologies, as well as efficient and reliable diagnostic methods.

### REFERENCES

Zhu X, Guo J, He C, Geng H, Yu G, Li J, Zheng H, Ji X, Yan F. Ultrasound triggered image-guided drug delivery to inhibit vascular reconstruction via paclitaxel-loaded microbubbles. Sci Rep. 2016 Feb. 22; 6:21683. doi: 10.1038/srep21683. PMID: 26899550; PMCID: PMC4761943.
Fan C H, Ting C Y, Liu H L, Huang C Y, Hsieh H Y, Yen T C, Wei K C, Yeh C K. Antiangiogenic-targeting drug-loaded microbubbles combined with focused ultrasound for glioma treatment. Biomaterials. 2013 March; 34(8):2142-55. doi: 10.1016/j.biomaterials.2012.11.048. Epub 2012 Dec. 14. PMID: 23246066.
Delalande A, Bastié C, Pigeon L, Manta S, Lebertre M, Mignet N, Midoux P, Pichon C. Cationic gas-filled microbubbles for ultrasound-based nucleic acids delivery. Biosci Rep. 2017 Dec. 22; 37(6): BSR20160619. doi: 10.1042/BSR20160619. PMID: 29180378; PMCID: PMC5741830.
Fan C H, Chang E L, Ting C Y, Lin Y C, Liao E C, Huang C Y, Chang Y C, Chan H L, Wei K C, Yeh C K. Folate-conjugated gene-carrying microbubbles with focused ultrasound for concurrent blood-brain barrier opening and local gene delivery. Biomaterials. 2016 November; 106:46-57. doi: 10.1016/j.biomaterials.2016.08.017. Epub 2016 Aug. 12. PMID: 27544926.

1. Lipid microbubble, comprising at least one cationic compound selected from lipophosphoramidates, histidylated polyethylenimines, and any mixture thereof.

2. Microbubble according to claim 1, further comprising at least one agent selected from a therapeutic agent, a targeting agent, a marking agent, and any combination thereof; said at least one agent preferably being:
i. exposed to the surface of the microbubble, or
ii. embedded in the microbubble's lipid envelope, or
iii. incorporated inside the microbubble, or
iv. any combination of i to iii.

3. Microbubble according to claim 1, characterized in that said at least one agent is selected from:
1) a nucleic acid;
2) a liposoluble active ingredient;
3) a chemotherapeutic agent, such as a cytotoxic agent and/or a cytostatic agent;
4) an antibody;
5) a protein;
6) an antigen;
7) a toxin;
8) a receiver;
9) an enzyme;
10) a hormone;
11) a ligand;
12) A viral vector;
13) a nanoparticle, preferably comprising at least one agent selected from a therapeutic agent, a targeting agent, a marking agent, and any combination thereof;
14) any derivative of 1) to 13), preferably any functional derivative thereof;
15) any fragment from 1) to 14), preferably any functional fragment thereof; and
16) any combination of 1) to 15).

4. Microbubble according to claim 1, further comprising at least one functional group enabling binding to at least one agent selected from a therapeutic agent, a targeting agent, a labelling agent, and any combination thereof; said group preferably being:
i. exposed to the surface of the microbubble, or
ii. embedded in the microbubble's lipid envelope, or
iii. incorporated inside the microbubble, or
iv. any combination of i to iii.

5. Microbubble according to claim 4, the functional group being selected from:
a peptide label;
a chemical group, preferably selected from a clickable function, a coupling group, and any combination thereof;
an antibody;
an antibody derivative;
a functional fragment of an antibody or antibody derivative;
an affinity label; and
any combination of these.

6. Microbubble according to claim 1, characterized in that the lipophosphoramidate is selected from the group consisting of a dimyristoyl phosphoramidate, preferably selected from a dimyristoyl bromide phosphoramidate, a dimyristoyl histamine phosphoramidate, and any combination thereof; a dioleyl phosphoramidate, preferably chosen from a dioleyl methylimidazolium phosphoramidate, and any combination thereof; a dipalmitoyl phosphoramidate; a disteraoyl phosphoramidate; and any mixture thereof.

7. Microbubble according to claim 1, characterized in that the histidylated polyethylenimine is coupled to a fatty acid, the fatty acid preferably being chosen from stearic acid, myristic acid, palmitic acid, oleic acid, and any combination thereof.

8. Microbubble according to claim 1, further comprising an additional lipid selected from the group consisting of dimyristoyl-glycero-phosphocholine, distearoyl-glycero-phosphocholine, dimyristoyl-glycero-phosphoethanolamine-polyethylene glycol, distearoyl-glycero-phosphoethanolamine-polyethylene glycol, and distearoyl-glycero-phosphoethanolamine-[biotinyl(polyethylene glycol)], cholesterol, beta-sitosterol, 1,2-dioleoyl-sn-glycero-3-phosphoethanolamine (DOPE), 1-oleoyl-2-[6-[(7-nitro-2-1,3-benzoxadiazol-4-yl)amino]hexanoyl]-3-trimethylammonium propane (DOTAP), a 1,2-dimyristoyl-rac-glycero-3-methoxypolyethylene glycol-2000 (DMG-PEG2000), and any combination thereof.

9. Microbubble according to claim 1, containing a biocompatible gas, the gas preferably being selected from perfluorobutane (C4F10), perfluoropropane (C3F8), dinitrogen (N2), sulfur hexafluoride (SF6), nitrogen oxide (NO), hydrogen, dioxygen, helium, xenon, argon, nitrous oxide (N2O), and any mixture thereof.

10. Pharmaceutical composition comprising at least one microbubble according to claim 1, and, optionally, a pharmaceutically acceptable excipient, the concentration of microbubbles in the composition preferably ranging from 10⁶ to 10¹⁴ microbubbles/ml, more preferably from 10⁷ to 10¹³ microbubbles/ml, more preferably from 10⁸ to 10¹² microbubbles/ml, more preferably from 10⁹ to 10¹¹ microbubbles/ml, more preferably the concentration of microbubbles in the composition being about 10¹⁰ microbubbles/ml.

11. Kit, including:
a) at least one microbubble according to claim 1, in a first container;
b) at least one therapeutic agent, in a second container;
c) optionally, at least one targeting agent in a third container;
d) optionally, at least one marking agent in a fourth container;
e) optionally, instructions for preparation and/or use;
the therapeutic agent and/or the targeting agent and/or the marking agent being preferably selected from:
1) a nucleic acid;
2) a liposoluble active ingredient;
3) a chemotherapeutic agent, such as a cytotoxic agent and/or a cytostatic agent;
4) an antibody;
5) a protein;
6) an antigen;
7) a toxin;
8) a receiver;
9) an enzyme;
10) a hormone;
11) a ligand;
12) a viral vector;
13) a nanoparticle, preferably comprising at least one agent selected from a therapeutic agent, a targeting agent, a marking agent, and any combination thereof;
14) any derivative of 1) to 13), preferably any functional derivative thereof;
15) any fragment from 1) to 14), preferably any functional fragment thereof; and
16) any combination of 1) to 15);
more preferably from a nucleic acid, a liposoluble active ingredient, a chemotherapeutic agent, an antibody, an antibody derivative, a functional fragment of an antibody or its derivative, a protein, a protein fragment, a nanoparticle, and any combination thereof.

12. Microbubble according to claim 1 for use as a medicament or as a marking agent.

13. A method of producing at least one microbubble according to claim 1, comprising the following steps:
a) A mixture, in a container, of cationic compounds selected from lipophosphoramidates, histidylated polyethylenimines, and any mixtures thereof; ethanol; and optionally at least one agent selected from a therapeutic agent, a targeting agent, a marking agent, and any combination thereof;
b) Evaporation of the mixture obtained in step a) to obtain a lipid film and rehydration of the lipid film to form a liposomal suspension;
c) Lyophilization of the liposomal suspension obtained in step b);
d) Replacement of the air contained in the container containing the lyophilizate obtained in step c), by a biocompatible gas, the gas preferably being selected from perfluorobutane (C4F10), perfluoropropane (C3F8), dinitrogen (N2), sulfur hexafluoride (SF6), nitrogen oxide (NO), hydrogen, dioxygen, helium, xenon, argon, nitrous oxide (N2O), and any mixture thereof;
e) Rehydration of the lyophilizate from step d) to obtain a solution;
f) Stirring the solution obtained in step d) to form microbubbles;
g) Optionally, functionalization of the microbubbles with at least one agent selected from a therapeutic agent, a targeting agent, a marking agent, and any combination thereof; and/or functionalization of the microbubbles with the addition of at least one functional group enabling binding to at least one agent selected from a therapeutic agent, a targeting agent, a marking agent, and any combination thereof.

14. A pharmaceutical composition according to claim 10 for use as a medicament or as a marking agent.

15. A kit according to claim 11 for use as a medicament or as a marking agent.
